(19) United States
(12) Patent Application Publication
(10) Pub. No.: US 2024/0390139 A1
(43) Pub. Date: Nov. 28, 2024

(54) COMMISSURAL ALIGNMENT BALLOON FOR TRANSCATHETER PROCEDURES

(71) Applicant: Anteris Technologies Corporation, Eagan, MN (US)

(72) Inventor: Christopher William Hummel, Minneapolis, MN (US)

(21) Appl. No.: 18/674,597

(22) Filed: May 24, 2024

Related U.S. Application Data
(60) Provisional application No. 63/469,267, filed on May 26, 2023.

(51) Int. Cl. A61F 2/24

(52) U.S. Cl. CPC A61F 2/2433; A61F 2/2418

(57) ABSTRACT

A balloon catheter system for delivering a prosthetic heart valve to a targeted site at a native valve of a patient includes a delivery shaft and a balloon fixedly attached to the delivery shaft. When the balloon is in a deflated state, a membrane material of the balloon defines a plurality of folded portions extending along a longitudinal axis of the delivery shaft. The balloon catheter system also includes a prosthetic heart valve disposed over the balloon when the balloon is in the deflated state and the prosthetic heart valve is in a collapsed state, a first set of folded portions of the plurality of folded portions extend clockwise about the longitudinal axis of the delivery shaft and a second set of folded portions of the plurality of folded portions extend counterclockwise about the longitudinal axis of the delivery shaft.

### CLAIM OF PRIORITY

This application claims the benefit of U.S. Provisional Patent Application No. 63/469,267 filed on May 26, 2023, the entire content of which is incorporated herein by reference.

### TECHNICAL FIELD

This document relates to delivery systems for medical devices and methods for their use. For example, this document relates to delivery systems for implantable medical devices such as prosthetic heart valves that are deliverable in a minimally invasive manner using a balloon catheter.

### BACKGROUND

Some prosthetic heart valves can be delivered in a minimally invasive fashion to avoid open-heart surgery. Such prosthetic heart valves can be delivered using a system of catheters that are manipulated by a clinician using an actuator handle and/or other types of control mechanisms that remain positioned external to the patient. For example, in some such cases, a prosthetic heart valve and a balloon are compressed into a balloon catheter, which may be expanded using one or more controls on the actuator handle.

Transcatheter aortic valve replacement (TAVR) delivery systems can be used to deliver a prosthetic aortic valve to a native aortic heart valve site. Clinicians occasionally encounter difficulty when delivering prosthetic aortic valves in a minimally invasive manner using such catheter-based delivery systems. One such area of difficulty pertains to the task of aligning the prosthetic heart valve with a native valve of the patient.

### SUMMARY

This document describes delivery systems for medical devices and methods for their use. For example, this document describes delivery systems for implantable medical devices such as, but not limited to, prosthetic heart valves that are deliverable in a minimally invasive manner using a system of catheters. In some embodiments, the delivery systems include a prosthetic heart valve that is disposed on a balloon catheter. The balloon catheter can be intravascularly advanced to a targeted treatment site of a patient, such as a native heart valve region (e.g., an aortic valve or other native heart valve) while under visualization from one or more imaging devices (e.g., a computed tomography (CT) imaging device, X-ray fluoroscopic system, etc.). This allows a clinician to navigate the balloon catheter to the targeted treatment site and to deploy the prosthetic heart valve in a desired alignment with anatomical features of the native heart valve.

To deploy the prosthetic heart valve, a balloon of the balloon catheter can be inflated, thus causing the prosthetic heart valve to transition from a low-profile radially collapsed state to an expanded state in which the prosthetic heart valve is operable. In some embodiments, before the balloon is inflated, the prosthetic heart valve is positioned by a clinician (e.g., an interventional cardiologist, surgeon, etc.) in a desired alignment and/or orientation relative to particular anatomical features of the native heart valve so that certain features of the prosthetic heart valve align with corresponding features of the native heart valve. For example, commissures and/or prosthetic leaflets of the prosthetic heart valve can be aligned with native commissures and/or leaflets of the native heart valve. It can be beneficial to align the prosthetic heart valve with the native heart valve when it is deployed at the targeted treatment site so that the prosthetic heart valve functions in a manner that is similar to the normal function of the heart valve.

In some embodiments, the prosthetic heart valve can be radially crimped to a collapsed state on a deflated balloon that is attached to the balloon catheter. When the prosthetic heart valve is in the collapsed state, the prosthetic heart valve defines a lumen that is smaller than a diameter of the balloon when inflated. This means that for the balloon to fit within the lumen of the collapsed valve, the deflated balloon can be folded or pleated so that the entire membrane material of the balloon fits within the lumen of the collapsed valve. As the balloon is inflated, the membrane material of the balloon expands outward, putting pressure on the inner surface of the valve. This causes the valve to expand, thus increasing a diameter of the lumen defined by the valve. As the balloon inflates, the pleats and/or deform, and the balloon eventually achieves a round cross section without pleats and/or folds.

In some cases, the alignment of the prosthetic heart valve to the native heart valve can change as the prosthetic heart valve transitions from the collapsed state to the expanded state. For example, the balloon of some balloon catheters, because of the particular way the balloon is folded or pleated within the collapsed prosthetic heart valve, apply rotational force to the prosthetic heart valve as it expands in response to the inflation of the balloon. This can cause the prosthetic heart valve to rotate relative to the native heart valve and to change the prosthetic heart valve's alignment relative to the native heart valve in an undesired manner. This means that it is beneficial to limit an amount that the valve rotates during expansion so that the clinician can align the valve properly prior to inflating the balloon without the alignment changing significantly as the balloon inflates. As described herein, an example system can include a balloon that is pleated and folded in a particular manner so that the balloon does not apply a rotational force to the prosthetic heart valve when it expands. Accordingly, using the balloon folding arrangements and techniques described herein, the alignment of the prosthetic valve to the native valve prior to deployment of the prosthetic valve is the same or closely similar to the alignment of the prosthetic valve to the native valve after deployment of the prosthetic valve.

In one aspect, a balloon catheter system includes a delivery shaft and a balloon fixedly attached to the delivery shaft. When the balloon is in a deflated state, a membrane material of the balloon defines a plurality of folded portions extending along a longitudinal axis of the delivery shaft. The balloon catheter system also includes a prosthetic heart valve disposed over the balloon when the balloon is in the deflated state and the prosthetic heart valve is in a collapsed state, a first set of folded portions of the plurality of folded portions extend clockwise about the longitudinal axis of the delivery shaft and a second set of folded portions of the plurality of folded portions extend counterclockwise about the longitudinal axis of the delivery shaft.

In another aspect, a balloon catheter for delivering a prosthetic heart valve to a targeted site at a native valve of a patient includes a delivery shaft and a balloon fixedly attached to the delivery shaft. When the balloon is in a deflated state, a membrane material of the balloon defines a plurality of folded portions extending along a longitudinal axis of the delivery shaft. When the balloon is disposed within a prosthetic heart valve when the balloon is in the deflated state and the prosthetic heart valve is in a collapsed state, a first set of folded portions of the plurality of folded portions extend clockwise about the longitudinal axis of the delivery shaft and a second set of folded portions of the plurality of folded portions extend counterclockwise about the longitudinal axis of the delivery shaft.

In another aspect, a method of pleating a balloon attached to a delivery shaft includes inflating the balloon such that a cross-section of the balloon is substantially circular and moving a set of pleating heads radially inward, each pleating head of the set of pleating heads pushing a membrane material of the balloon inwards to define a recess of a set of recesses. Each pleating head comprises a pleating tooth located at a distal end of the respective pleating head. The method also includes placing a pleating nut to secure the pleating tooth of each pleating head, preserving the set of recesses and retracting the set of pleating heads radially outward, leaving the pleating tooth corresponding to each pleating head secured to the pleating nut. The method also includes moving a set of folding heads radially inwards, each folding head of the set of folding heads pushing a membrane material of the balloon inwards to define a plurality of folded portions and arranging the plurality of folded portions so that a first set of folded portions of the plurality of folded portions extend clockwise about a longitudinal axis of the delivery shaft and a second set of folded portions of the plurality of folded portions extend counterclockwise about the longitudinal axis of the delivery shaft.

Particular embodiments of the subject matter described in this document can be implemented to realize one or more of the following advantages.

In some embodiments, the deflated balloon is pleated and folded to fit within the lumen of a prosthetic heart valve that is then radially crimped onto the balloon. For example, the membrane of the deflated balloon can be folded over itself to form folded-over portions, with the folded-over portions running parallel to a longitudinal axis of the balloon catheter. These folded-over portions can be arranged to extend in both rotational directions (e.g., both counterclockwise and clockwise) about the longitudinal axis of the balloon catheter and so that the folded-over portions are not all extending in the same direction. When the folded-over portions are extending or deflected both clockwise and counterclockwise, this allows the balloon to be inflated while preventing or substantially limiting rotations of the prosthetic valve that may otherwise be induced by the balloon as it is expanded. This improves an ability of a clinician to align the prosthetic heart valve with the native valve prior to inflating the balloon and expanding the valve as compared with prosthetic heart valve delivery systems where folds are extending or deflected in the same direction (e.g., all clockwise, all counterclockwise, a majority clockwise, or a majority counterclockwise). Since the equal number of opposingly extending folded-over portions facilitate the alignment of the prosthetic heart valve to stay substantially the same relative to the native heart valve as the prosthetic heart valve expands, this allows the clinician to align certain features of the prosthetic heart valve (e.g., commissure posts, leaflets, etc.) with corresponding features of the native valve (e.g., commissures, leaflets, etc.) prior to expanding the balloon. The prosthetic heart valve maintains this alignment upon expansion/deployment.

In some embodiments, a pleating and folding device is advantageously configured to form pleats and folds in the deflated balloon. For example, the pleating device can include a set of fold heads and a set of pleat heads that compress the outer surface of the balloon inwards to form a set of folds. The folds each represent a portion of the deflated balloon membrane that folds over itself. The folds of the balloon can be arranged so that the balloon fits within a lumen of the collapsed prosthetic heart valve. As the balloon is inflated, the folds of the balloon fill with fluid and deform, causing the balloon to apply pressure to the inner walls of the prosthetic heart valve. As the balloon expands outward, this transitions the balloon from the collapsed state to the expanded state while limiting a rotation of the prosthetic heart valve to less than a threshold amount of rotation.

The balloon, in some embodiments, can form one or more advantageous patterns while deflated that ultimately cause the prosthetic heart valve to expand radially outward while limiting rotation as the balloon is inflated. For example, the pleats can be formed by creating folds of the membrane of the balloon. A fold is a portion of the membrane that is folded over such that the membrane makes a reverse loop to come in contact with itself. These folds can include one or more folds deflected in a first direction (e.g., extending clockwise about the longitudinal axis of the balloon) and one or more folds deflected in a second direction (e.g., extending counterclockwise about the longitudinal axis of the balloon) while the deflated balloon is disposed within a lumen of the prosthetic heart valve. As the balloon inflates, the folds deflected in the first direction exert outward radial forces and tangential forces and the folds deflected in the second direction exert outward radial forces and tangential forces. The tangential forces of the folds deflected in opposite directions cancel each other and the outward radial forces of the folds deflected in opposite directions work together to radially expand the prosthetic heart valve. Consequently, the balloons that are folded in the patterns described herein provide improved techniques and results for expanding a prosthetic heart valve. That is, the balloons that are folded in the patterns described herein decrease an amount of rotation of the valve as the valve expands as compared with balloon catheters where the deflated balloon has folds that are all deflected to extend in the same direction.

The pleated balloon can form one or more open pleats that are defined by folds of the membrane to form an open recess. In some embodiments, these open recesses and closed recesses are linear and run parallel to the longitudinal axis of the balloon. This means that as the balloon inflates and the folds deform, the recesses expand radially outward. Since the recesses are linear, a rotation of the material of the balloon is limited, thus limiting a rotation of the prosthetic heart valve to less than a threshold amount of rotation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF DRAWINGS

FIG. 1 is a perspective view of an example medical device delivery system in accordance with some embodiments provided herein.

FIG. 2 is an enlarged perspective view of an example distal end portion of the medical device delivery system of FIG. 1.

FIG. 3 is an enlarged perspective view of an example handle of the medical device delivery system of FIG. 1.

FIG. 4 is a cutaway view from a cutaway location of the example distal end portion of the medical device delivery system of FIG. 1.

FIG. 5 is a plan view of the distal end portion of the medical device delivery system of FIG. 1 in a delivery configuration.

FIG. 6 is a plan view of the distal end portion of the medical device delivery system of FIG. 1 in an intermediate configuration.

FIG. 7 is a plan view of the distal end portion of the medical device delivery system of FIG. 1 in an expanded configuration.

FIG. 8A is a perspective view of the distal end portion of the medical device delivery system of FIG. 1 without the implantable medical device.

FIG. 8B is a perspective view of an example valve stop member in accordance with some embodiments.

FIG. 9A is a diagram illustrating an enlarged version of the cutaway view of the balloon catheter of FIG. 4, the enlarged version indicating one or more forces applied by the balloon to the prosthetic heart valve.

FIG. 9B is a diagram illustrating a cutaway perspective view of the balloon arranged according to the pattern illustrated in FIG. 9A to fit within a lumen of the collapsed prosthetic heart valve.

FIG. 10 is a diagram of a set of cutaway locations of a balloon.

FIGS. 11-18 illustrate cutaway views of a balloon in the deflated state while disposed within the lumen of a prosthetic heart valve in the compressed state at each of the cutaway locations of FIG. 10.

FIG. 19 illustrates a pleating tool for pleating a balloon to fit within the recess of a prosthetic heart valve in the collapsed state.

FIG. 20 illustrates a pleating tool for pleating a balloon in a second pleating position where pleating heads and pleating teeth are extended inwards to create a set of cavities.

FIG. 21 illustrates a pleating tool for pleating a balloon in a third pleating position where pleating heads are retreated and pleating teeth preserve cavities in the balloon and a pleating nut is attached to secure pleating teeth in place.

FIG. 22 illustrates a pleating tool for pleating a balloon in a fourth pleating position where pleating heads are retreated and pleating teeth preserve cavities in the balloon while secured by a pleating nut.

FIG. 23 illustrates a pleating tool for pleating a balloon in a fifth pleating position where folding heads are advanced to create folded-over portions in the membrane of the balloon.

FIG. 24 illustrates pleating tool for pleating a balloon in a sixth pleating position where pleating heads and folding heads are retracted and the folded-over portions in the membrane of the balloon are preserved by vacuum.

FIG. 25 is a flow diagram illustrating an example operation for expanding a balloon catheter including a pleated balloon with linear recesses.

### DETAILED DESCRIPTION

This document relates to delivery systems for medical devices and methods for their use. For example, this document relates to delivery systems for implantable medical devices such as prosthetic heart valves that are deliverable in a minimally invasive manner using a balloon catheter. In some embodiments, the delivery systems include a balloon catheter that includes a balloon attached to a distal end portion of the balloon catheter, and a prosthetic heart valve disposed over the balloon in a radially crimped or collapsed state. When the balloon is deflated, the membrane material of the balloon can be pleated and folded into a particular pattern that can be expanded while limiting rotational force applied to the prosthetic heart valve, as described further herein. For example, the deflated balloon can include a plurality of folded-over portions that extend in opposing directions about a longitudinal axis of the balloon. This ensures that as the folded-over portions radially expand and unfold, the folded-over portions do not apply a net rotational force to the prosthetic heart valve that causes the valve to rotate as the valve is being radially expanded.

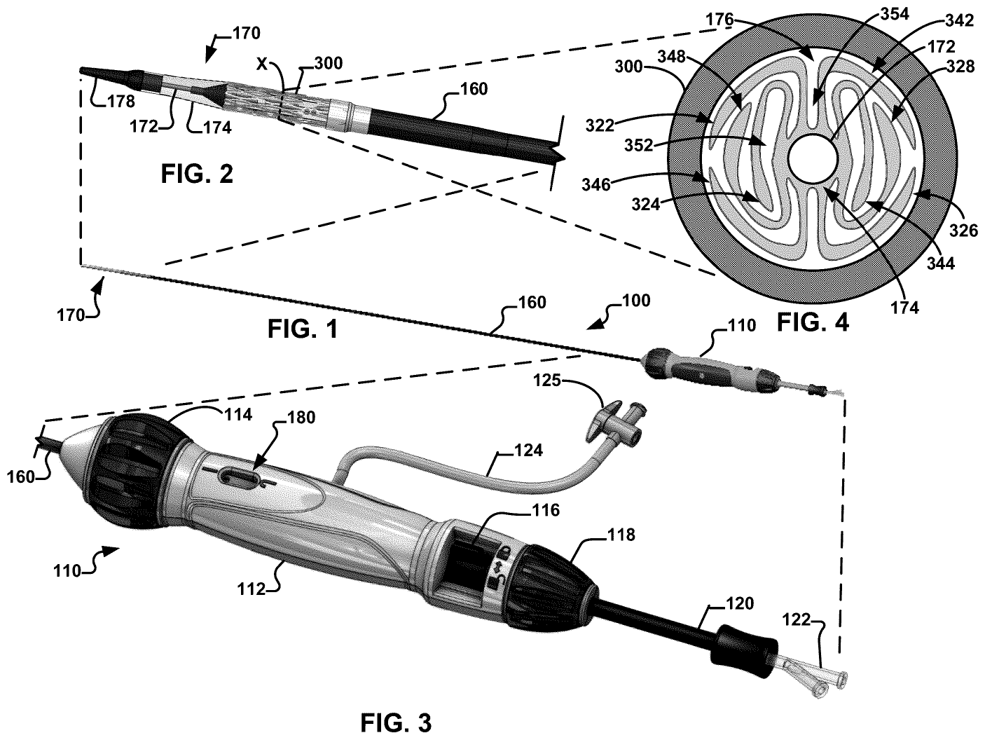

FIG. 1 illustrates an example transcatheter medical device delivery system 100. In the depicted example, the medical device delivery system 100 is configured to deliver a prosthetic heart valve to a native heart valve location by advancing the prosthetic heart valve to the heart via the vascular system of the patient. For example, in some embodiments the medical device delivery system 100 can be used to deliver a prosthetic aortic valve to a site of a native aortic valve via the aorta of the patient. This non-limiting type of use of the medical device delivery system 100 is used as an example herein to describe the functionality of the medical device delivery system 100. In such a case, the medical device delivery system 100 may be inserted into a femoral artery via a sheath and then advanced to the aorta, through the aortic arch, and to the native aortic valve site. Alternative approaches, such as trans-subclavian, trans-carotid, trans-radial, and others are also envisioned using the medical device delivery system 100.

Broadly speaking, the medical device delivery system 100 includes a clinician control handle 110 (or simply “handle 110”), a steerable catheter 160, and an elongate catheter 170 (referred to hereinafter as a balloon catheter 170). The steerable catheter 160 and the balloon catheter 170 each extend distally from the handle 110. The steerable catheter 160 and the balloon catheter 170 are each affixed to the handle 110, but at different locations of the handle 110 (as described further below).

In some embodiments, the medical device delivery system 100 is configured to deliver the prosthetic heart valve to the native heart valve location under visualization from one or more medical imaging systems. These medical imaging systems can include X-ray systems, computed tomography (CT) systems, or other kinds of medical imaging systems. This visualization allows a clinician to view the location of medical device delivery system 100 relative to the site of the native aortic valve and the orientation of the prosthetic heart valve relative to the native aortic valve, among other things.

In some embodiments, balloon catheter 170 and a distal portion of steerable catheter 160 may be inserted into the vascular system of the patient while the handle 110 remains outside of the patient. A clinician can control the steerable catheter 160 and the balloon catheter 170 by guiding the medical device delivery system 100 further into the patient, retracting the medical device delivery system 100 from the patient, and using one or more actuators on the handle 110. The FIG. 2 shows an expanded view of a distal end portion of the steerable catheter 160 and the balloon catheter 170. An example balloon 174 and an example prosthetic heart valve 300 are mounted on the balloon catheter 170 in a low-profile delivery configuration.

The steerable catheter 160 defines a lumen in which the balloon catheter 170 is slidably disposed. That is, the balloon catheter 170 can be manipulated by the clinician (using the handle 110) to advance and/or retract the balloon catheter 170 (and the prosthetic heart valve 300) relative to the steerable catheter 160 by sliding the balloon catheter 170 within the lumen of the steerable catheter 160. For example, in some cases the steerable catheter 160 can be proximally pulled back relative to the balloon catheter 170 and the prosthetic heart valve 300, as described further below.

The steerable catheter 160 is controllably deflectable or steerable by the clinician (using the handle 110 to manipulate a pull wire, as described further below in reference to FIG. 3). In particular, a distal end portion of the steerable catheter 160 is controllably deflectable to any desired angle up to approximately 180°, or even more than 180° in some embodiments. When the steerable catheter 160 is deflected in that manner, the balloon catheter 170 also takes on the same extent of deflection (because the balloon catheter 170 is positioned within the lumen of the steerable catheter 160). The deflection of the steerable catheter 160 (and the balloon catheter 170) can be useful for navigating the aortic arch, for example. In some embodiments, the balloon catheter 170 (and the steerable catheter 160) can be advanced over a pre-placed guidewire.

Still referring to FIG. 2, the balloon catheter 170 includes an inner catheter shaft 172 and a balloon 174 mounted on a distal end portion of the balloon catheter 170. The inner catheter shaft 172 defines an inflation lumen and one or more openings (not visible) through which an inflation fluid can be supplied and withdrawn in order to controllably inflate and/or deflate the balloon 174. In addition, the inner catheter shaft 172 defines a central lumen by which the balloon catheter 170 (and the steerable catheter 160) can be advanced over a guidewire.

In the depicted embodiment, a tapered nose cone 178 is attached to a distal end of the inner catheter shaft 172 and to the balloon 174. The tapered nose cone 178 extends distally from the balloon 174 and provides an atraumatic leading distal end of the medical device delivery system 100. In some examples, the tapered nose cone 178 may have a rounded distal end that does not include any sharp edges so that balloon catheter 170 can navigate through the vasculature of the patient without puncturing or otherwise damaging tissue of the patient. In some embodiments, a surface of the nose cone angles gently inwards towards the longitudinal axis of balloon catheter 170 so that a diameter of tapered nose cone 178 decreases gradually moving distally along tapered nose cone 178. In some cases, this gradual tapering of tapered nose cone 178 allows nose cone to gently increase a diameter of narrow passageways in the patient's anatomy to advance balloon catheter 170 through the vasculature without causing damage to tissue.

The prosthetic heart valve 300 can be radially crimped onto the balloon 174 to reside thereon in a radially compressed, low-profile delivery configuration. When the crimped prosthetic heart valve 300 is on the balloon 174 in preparation for deployment, the prosthetic heart valve 300 is in a collapsed state and the balloon 174 is in a deflated state. In some embodiments, the prosthetic heart valve 300 includes a metallic stent frame. In some examples, the frame comprises one piece of material that is configured to transition from the collapsed state to the expanded operable state in response to the balloon 174 being expanded from the deflated state to an inflated state.

As described in further detail below, the frame of prosthetic heart valve 300 can include a set of commissure posts. In some cases, these commissure posts can be analogous to commissures of a native heart valve. For example, prosthetic heart valve 300 can include three commissures, which is the same number of commissures of most native aortic valves. In some embodiments, the set of prosthetic heart valve leaflets of prosthetic heart valve 300 can include three prosthetic heart valve leaflets, which is the same number of leaflets of most native aortic valves. The frame of prosthetic heart valve 300 can, in some embodiments, include a set of midpoint connectors, where each midpoint connector of the set of midpoint connectors is spaced between each pair of consecutive commissure posts. In embodiments where the frame includes three commissure posts, the frame can include three midpoint connectors.

In some embodiments, the prosthetic heart valve leaflets of prosthetic heart valve 300 can be attached to the frame at the commissure posts of the frame. For example, each prosthetic heart valve leaflet can be attached to two commissure posts, one commissure post at either end of the leaflet. In some embodiments, the prosthetic heart valve 300 includes three commissure posts and three prosthetic leaflets corresponding to the three commissures and three leaflets of most native aortic valves. It may be beneficial to align an orientation of the commissure posts and the prosthetic valve leaflets of the prosthetic heart valve 300 with an orientation of commissures and the leaflets of the native aortic heart valve. This can involve rotating prosthetic heart valve 300 relative to the native aortic heart valve such that when prosthetic heart valve 300 is expanded, each of the commissure posts of the frame occupies a rotational position that is similar to the rotational position of the corresponding native aortic commissure and each prosthetic heart valve leaflet of prosthetic heart valve 300 occupies a rotational position that is similar to the rotational position of a corresponding native aortic leaflet.

The balloon 174 in the deflated state is sized to fit within a lumen of the prosthetic heart valve 300 in the collapsed state such that a greatest diameter of the balloon 174 in the deflated state is less than or equal to the diameter of the lumen of the prosthetic heart valve 300 in the collapsed state. For example, the balloon 174 can include a membrane comprising a malleable material such as nylon, polyurethane, polyethylene terephthalate (PET), or another material. When the balloon 174 is in a deflated state, this membrane material can be manipulated into a pleated pattern so that balloon 174 fits within a lumen of the collapsed prosthetic heart valve 300, even when the diameter of the lumen is smaller than a diameter of the balloon when inflated.

The pleated and folded pattern of balloon 174 within the lumen of the collapsed prosthetic heart valve 300 can, in some embodiments, define a plurality of recesses. For example, when the balloon 174 is in a deflated state within the lumen of the collapsed prosthetic heart valve 300, the balloon can be pleated and folded to arrange excess membrane material so that the cross-section of the balloon 174 is small enough to fit within the lumen of the collapsed prosthetic heart valve 300. Folding over excess membrane material can result in a plurality of folds and/or folded-over portions. This plurality of folds and/or folded-over portions can define one or more recesses. In some embodiments, recesses defined by the membrane material of the balloon 174 within the lumen of the collapsed prosthetic heart valve 300 are substantially linear and parallel to a longitudinal axis of the inner catheter shaft 172 of the balloon catheter 170.

To arrange the folded portions of the deflated balloon 174 so that the balloon 174 fits within the lumen of the collapsed prosthetic heart valve 300, the folded portions can be deflected rotationally about a longitudinal axis of the balloon catheter 170. For example, before being deflected, each folded portion of the plurality of folded portions can extend radially outward relative to the longitudinal axis of balloon catheter 170. One or more of the folded portions can be deflected or wrapped clockwise about the longitudinal axis of the balloon catheter 170 and one or more folded portions can be deflected or wrapped counterclockwise about the longitudinal axis of the balloon catheter 170 so that a maximum diameter of a cross-section of the deflated balloon 174 is less than or equal to a diameter of the collapsed prosthetic heart valve 300. When equal numbers of folded portions are deflected or wrapped to extend about the longitudinal axis of the balloon catheter 170 in different/opposing directions (clockwise versus counterclockwise), this causes the folded-over portions to apply opposing rotational forces to the prosthetic heart valve 300 during expansion that cancel out each other to thereby prevent or substantially limit rotation of the prosthetic heart valve 300 during its expansion.

In some embodiments, balloon 174 can be rotated about a longitudinal axis of balloon catheter 170 when balloon 174 is in the collapsed state. This means that when balloon catheter 170 is inserted inside of the patient, balloon catheter 170 can be rotated. In some cases, when balloon catheter 170 rotates, prosthetic heart valve 300 and balloon 174 also rotates with balloon catheter 170. This means that in cases where prosthetic heart valve 300 is advanced to a targeted site of the patient such as the site of the native aortic valve, balloon catheter 170 can be rotated so that prosthetic heart valve 300 properly aligns with the native aortic valve. In some embodiments, balloon catheter 170 rotates relative to steerable catheter 160 such that steerable catheter 160 remains in place as balloon catheter 170 rotates. Since a shaft (e.g., inner catheter shaft 172 and/or an outer catheter shaft) of balloon catheter 170 can be slidably disposed within a lumen of steerable catheter 160, the shaft of balloon catheter 170 can rotate within the lumen of steerable catheter 160 to cause prosthetic heart valve 300 to rotate and without causing steerable catheter 160 to rotate.

As described further below, when the balloon 174 and the prosthetic heart valve 300 are positioned at a target location and in a desired orientation relative to the patient's anatomy, the balloon 174 can be inflated to radially expand the prosthetic heart valve 300 into engagement with the native anatomy of the patient (e.g., into engagement with the annulus of a native heart valve such as the native aortic valve). Thereafter, the balloon 174 can be deflated and retracted from the prosthetic heart valve 300. In some embodiments, radiopaque markers can be located on one or more locations of the steerable catheter 160, the balloon catheter 170, the prosthetic heart valve 300, or any combination thereof. This can provide visualization of the steerable catheter 160, the balloon catheter 170, and the prosthetic heart valve 300 under fluoroscopy so that the clinician can view a location of the steerable catheter 160, the balloon catheter 170, and the prosthetic heart valve 300 relative to the patient's anatomy. In some cases, radiopaque markers and/or other fiducial markers can provide a visualization of the orientation of prosthetic heart valve 300 relative to the native aortic valve.

When the balloon 174 is inflated, this causes the membrane material of the balloon 174 to expand outwards relative to the shaft of the balloon catheter 170, causing prosthetic heart valve 300 to also expand. For example, as balloon 174 transitions from the deflated state to the inflated state, this causes prosthetic heart valve 300 to expand from the collapsed state to the expanded state. During expansion of prosthetic heart valve 300, a diameter of prosthetic heart valve 300 can increase in response to the balloon 174 applying pressure to the inner wall of prosthetic heart valve 300 as balloon 174 inflates. This pressure applied by balloon 174, in some examples, includes one or more radial and/or rotational forces that are applied by folded portions of the balloon membrane as the folded portions deform or unfold in response to inflation. When the folded portions of the deflated balloon 174 are wrapped to extend in opposing directions about the longitudinal axis of balloon catheter 170 as the balloon 174 is disposed within the lumen of prosthetic heart valve 300, this can cause the folded portions of balloon 174 to apply opposing rotational forces that cancel each other so that a rotation of prosthetic heart valve 300 during expansion is limited to less than a threshold amount of rotation. For example, prosthetic heart valve 300 can expand radially outward as balloon 174 inflates. A difference between a rotational position of prosthetic heart valve 300 in the collapsed state and prosthetic heart valve 300 in the expanded state, in some embodiments, is less than a threshold amount of rotation. In some examples, this threshold amount of rotation is 5 degrees, 10 degrees, or 20 degrees.

FIG. 3 shows an enlarged view of an example handle 110 of the medical device delivery system 100. The handle 110 remains external to the patient while the steerable catheter 160, the balloon catheter 170, and the prosthetic heart valve 300 extend internally to the patient (e.g., into the vasculature and/or heart of the patient). The handle 110 includes multiple control mechanisms by which a clinician operator can remotely manipulate various aspects of the steerable catheter 160 and the balloon catheter 170, as described further below. The steerable catheter 160 is fixed to the handle 110.

In this view of the handle 110, the following components and/or control mechanisms of the handle 110 are in view. That is, the handle 110 includes a housing 112, a rotatable first actuator knob 114, a locking actuator 116, a rotatable second actuator knob 118, a balloon catheter pull rod 120, a fill line 122, a flush line 124, and an optional deflection indicator 180.

The first actuator knob 114, the second actuator knob 118, and the locking actuator 116 are each manually rotatable relative to the housing 112. The steerable catheter 160 can be laterally deflected by rotation of the first actuator knob 114. That is, manual rotations of the first actuator knob 114 can be used to control the extent of deflection of the steerable catheter 160 (and the balloon catheter 170 disposed therein) by tensioning or relaxing a pull wire (not shown). The deflection indicator 180 can provide an indication of the extent of deflection of the steerable catheter 160. The second actuator knob 118 can be rotated to rotate the balloon catheter 170 (and the prosthetic heart valve 300) relative to the steerable catheter 160.

A proximal end of the balloon catheter 170 is affixed to the balloon catheter pull rod 120. The balloon catheter pull rod 120 is manually translatable relative to the housing 112 (when the locking actuator 116 is in its unlocked position). The balloon catheter pull rod 120 can be extended and retracted relative to the housing 112 to extend and retract the balloon catheter 170 (and the prosthetic heart valve 300) relative to the steerable catheter 160. The locking actuator 116 can be used to lock and unlock the movability of the balloon catheter pull rod 120 relative to the housing 112.

In some examples, the fill line 122 may be configured to deliver fluid and remove fluid from the balloon 174 of the balloon catheter 170. For example, the fill line 122 can be connected to a fill lumen that extends through balloon catheter pull rod 120, steerable catheter 160, and balloon catheter 170 to reach the balloon 174. To inflate the balloon 174, fluid can be introduced via the fluid line 122 such that the fluid flows to an inside of the balloon 174. As the volume of fluid within balloon 174 increases, this causes the membrane of the balloon 174 to expand radially outward to accommodate the increasing volume of fluid within the balloon 174. This causes the balloon 174 to apply pressure to the inner surface of prosthetic heart valve 300, causing prosthetic heart valve 300 to radially expand. To deflate the balloon, a clinician operator can cause fluid to discharge through the fill line 122. Handle 110 can also include a flush line 124 including an actuator 125.

FIG. 4 shows a cross-sectional view of inner catheter shaft 172, balloon 174, and prosthetic heart valve 300 at a position X (FIG. 2) along the balloon catheter 170. In the embodiment of FIG. 4, prosthetic heart valve 300 is in a collapsed state and balloon 174 is deflated. In the collapsed state, prosthetic heart valve 300 defines a lumen 176. Balloon 174 is disposed within the lumen 176 and inner catheter shaft 172 longitudinally extends through a center of lumen 176.

Within the lumen 176 of prosthetic heart valve 300 in the collapsed state, the lumen of balloon 174 is formed in an example pattern that comprises a plurality of folded portions in some embodiments. As seen in FIG. 4, the plurality of folded portions (or simply “folds”) includes a first set of folds 322, 324, 326, and 328 (“322-328”) and a second set of folds 342, 344, 346, and 348 (“342-348”). In some embodiments, the first set of folds 322-328 are each deflected or wrapped to extend in a first rotational direction (e.g., counterclockwise in the depicted embodiment) about a longitudinal axis of balloon catheter 170 and the second set of folds 342-348 are each deflected in a second rotational direction (e.g., clockwise in the depicted embodiment) about the longitudinal axis of balloon catheter 170. In the example of FIG. 4, there are a total of eight folded portions. The first set of folds 322-328 includes four folds (i.e., fold 322, fold 324, fold 326, fold 328) and the second set of folds 342-348 includes four folds (i.e., fold 342, fold 344, fold 346, fold 348). In other embodiments, other total numbers of folds can be included, such as two folds, four folds, six folds, ten folds, twelve folds, and so on.

In embodiments where a number of folds of the balloon 174 deflected in the first direction is equal to a number of folds of the balloon 174 deflected in the second direction, rotational forces applied to the prosthetic heart valve 300 by the expanding the folds cancel each other such that a rotation of prosthetic heart valve 300 as the balloon 174 expands is substantially eliminated or is less than a threshold amount of rotation. For example, as balloon 174 expands, fold 322 may apply force to the inner surface of prosthetic heart valve 300 that extends radially outward and rotationally clockwise relative to the longitudinal axis of balloon catheter 170 since fold 322 is deflected in the counterclockwise direction. Fold 346 may apply force to the inner surface of prosthetic heart valve 300 that extends radially outward and rotationally counterclockwise relative to the longitudinal axis of balloon catheter 170 since fold 346 is deflected in the clockwise direction. The rotational forces applied by fold 322 and fold 346 can, in some embodiments, cancel each other such that these rotational forces do not cause substantial rotation of prosthetic heart valve 300. The radial forces that fold 322 and fold 346 apply to the inner surface of prosthetic heart valve 300 can cause the prosthetic heart valve 300 to expand from the collapsed state to the expanded state.

The first set of folds 322-328 and the second set of folds 342-348 can be formed when balloon 174 is pleated and folded to reside within the lumen 176 of the prosthetic heart valve 300 that is radially crimped thereon. Balloon 174 can inflate to a diameter that is greater than the diameter of the lumen 176 of prosthetic heart valve 300 in the collapsed state. This means that excess portions of the membrane of the balloon 174 are folded over each other and arranged to compress balloon 174 into a smaller diameter. In some embodiments, the balloon 174 is folded and arranged as shown in FIG. 4 but this is not the only possible arrangement of balloon 174. Balloon 174 can be folded and arranged in one or more other configurations that result in the balloon 174 having a diameter that fits within a lumen of prosthetic heart valve 300. These one or more configurations can include folds that are deflected in opposing directions so that rotational forces applied to prosthetic heart valve 300 during expansion cancel to limit a rotation of prosthetic heart valve 300. When balloon 174 is fully inflated, the membrane of balloon 174 does not include folds and the cross-section of the balloon 174 is round (e.g., circular). However, the diameter of the balloon 174 is significantly larger when balloon 174 is inflated as compared with when balloon 174 is deflated, pleated, and folded to fit within the lumen 176 of the collapsed prosthetic heart valve 300. Pleating and folding the deflated balloon 174 represents one beneficial way to reduce a greatest diameter of the balloon 174 to fit within the lumen 176.

In some embodiments, the folds (e.g., first set of folds 322-328 and second set of folds 342-348) of the balloon 174 are arranged within the lumen 176 of the collapsed prosthetic heart valve 300 to form one or more recesses. These recesses include closed recesses and open recesses. For example, fold 324 and fold 348 form a closed recess 352, because fold 324 and fold 348 bend inwards towards each other to form closed recess 352 as a closed channel. In some examples, closed recess 352 extends along balloon 174 substantially along the longitudinal axis of balloon catheter 170. Closed recess 352 is not the only closed recess formed by the membrane of balloon 174. Any gap formed by folds of the membrane that are deflected inwards to overlap represents a closed recess. Fold 322 and fold 354 form an open recess 354, because fold 322 and fold 354 bend away from each other such that open recess 354 is an open channel. In some examples, open recess 354 extends along balloon 174 substantially along the longitudinal axis of balloon catheter 170. Open recess 354 is not the only open recess formed by the membrane of balloon 174. Any open channel formed by a pair of folds tat at deflect away from each other is an open recess.

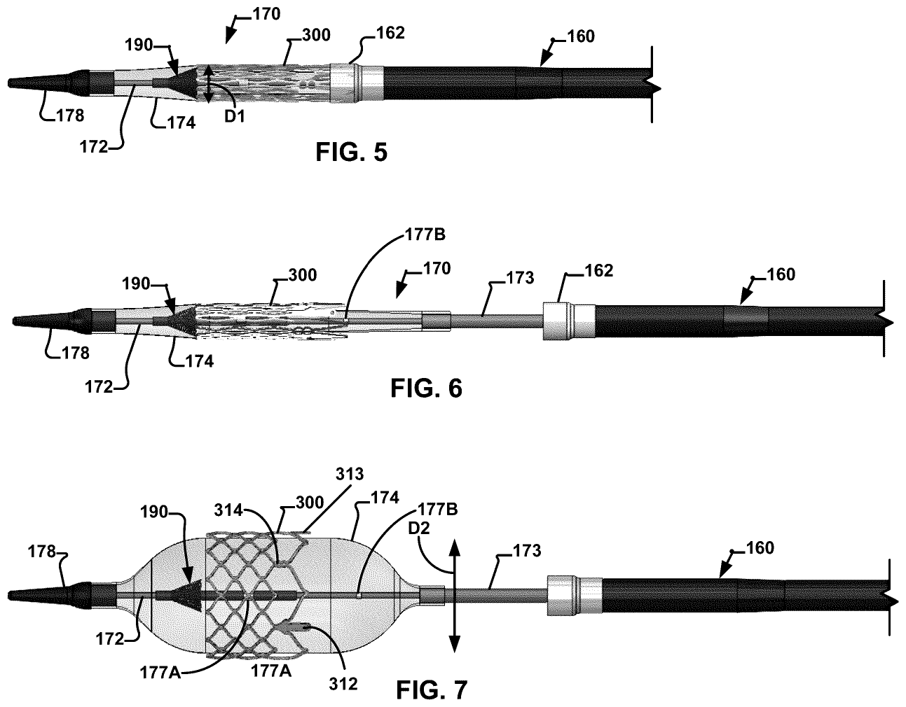

FIG. 5 provides another view of the distal end portion of the medical device delivery system 100, including the steerable catheter 160 and the balloon catheter 170. In the example of FIG. 5, the prosthetic heart valve 300 in the collapsed state is mounted on the balloon 174 in the deflated state. The steerable catheter 160 and the prosthetic heart valve 300 are attached to the balloon catheter 170. The tapered nose cone 178 and the inner catheter shaft 172 of the balloon catheter 170 are also visible. The configuration depicted in FIG. 5 is a delivery configuration that is used when advancing the distal end portion of the medical device delivery system 100 and the prosthetic heart valve 300 to a targeted site for deployment of the prosthetic heart valve 300 within a patient (e.g., to a native heart valve region). For example, the prosthetic heart valve 300 can be in the collapsed state during advancement to the targeted site so that a diameter of the distal end portion of the medical device delivery system 100 is small enough for the prosthetic heart valve 300 to navigate through the vasculature of the patient to the targeted site.

In addition, a valve stop member 190 is shown. The valve stop member 190 is attached to the inner catheter shaft 172 of the balloon catheter 170 and located within the balloon 174. The valve stop member 190 can engage with a distal end of the prosthetic heart valve 300 to prevent prosthetic heart valve 300 from advancing distally past valve stop member 190 when prosthetic heart valve 300 is in the collapsed state. This can help to hold prosthetic heart valve 300 in place as balloon catheter 170 advances to the targeted site of the patient. Furthermore, this can help to cause prosthetic heart valve 300 to rotate with balloon catheter 170 when balloon catheter 170 is rotated while prosthetic heart valve 300 is in the collapsed state. Additional features of the valve stop member 190 are described below.

In the depicted delivery configuration, the prosthetic heart valve 300 is longitudinally compressed in the collapsed state and securely captured between a flared distal end 162 of the steerable catheter 160 and the valve stop member 190. The flared distal end 162 defines an annular space that receives and covers an end portion of the prosthetic heart valve 300. That is, an end portion of the prosthetic heart valve 300 (e.g., of the metallic stent frame of the prosthetic heart valve 300) is concealed by the flared distal end 162 of the steerable catheter 160. This arrangement helps to prevent the potential for vessel wall damage that the end portion of the prosthetic heart valve 300 may otherwise incur if the end portion was exposed (rather than being concealed by the flared distal end 162 of the steerable catheter 160). Accordingly, during transvascular advancement of the depicted arrangement, the coverage of the end portion of the prosthetic heart valve 300 by the flared distal end 162 mitigates risks of vessel wall damage that could result if that end portion was to make contact with the vessel walls.

The other end of the prosthetic heart valve 300 is held in position by the valve stop member 190. The valve stop member 190 prevents the prosthetic heart valve 300 from moving distally despite the longitudinal force from the flared distal end 162 of the steerable catheter 160 that would otherwise engender such distal movement. Accordingly, the prosthetic heart valve 300 can be captured between the steerable catheter 160 and the valve stop member 190 when the prosthetic heart valve 300 is in the collapsed state. The flared distal end 162 and the valve stop member 190 can secure the prosthetic heart valve 300 in place so that the steerable catheter 160 and the balloon catheter 170 do not have any sharp leading edges. This allows balloon catheter 170 to advance through the vasculature of the patient to the targeted site without causing damage to the tissue of the patient.

Because the valve stop member 190 resides within the balloon 174, a layer of the flexible wall material of the balloon 174 resides between the prosthetic heart valve 300 and the valve stop member 190. That flexible wall material of the balloon 174 is compressed between the prosthetic heart valve 300 and the valve stop member 190 in the depicted delivery configuration.

FIG. 6 depicts a latter stage of the delivery/deployment process of the prosthetic heart valve 300 using the medical device delivery system 100. In comparison to the arrangement of FIG. 4, here the steerable catheter 160 has been pulled proximally back in relation to the balloon catheter 170 and/or the balloon catheter 170 has been pushed distally forward in relation to steerable catheter 160. As seen in the example of FIG. 6, when the balloon catheter 170 is displaced from the steerable catheter 160, the prosthetic heart valve 300 remains mounted in the collapsed state on the balloon 174 of the balloon catheter 170.

In some examples, the balloon catheter 170 can be pushed away from the steerable catheter 160 or pulled towards the steerable catheter 160 based on user control of the balloon catheter pull rod 120 illustrated in FIG. 3. For example, pulling the balloon catheter pull rod 120 proximally relative to steerable catheter 160 pulls the balloon catheter 170 towards steerable catheter 160 and pushing the balloon catheter pull rod 120 distally relative to steerable catheter 160 pushes the balloon catheter 170 away from steerable catheter 160. In some cases, the steerable catheter 160 and the balloon catheter 170 can separate from each other as seen in FIG. 6 when the prosthetic heart valve 300 is proximate to the targeted treatment site.

The depicted arrangement reveals that the balloon catheter 170 also includes an outer catheter shaft 173. The inner catheter shaft 172 extends distally from the outer catheter shaft 173. The proximal end of the balloon 174 is attached to a distal end portion of the outer catheter shaft 173. The distal end of the balloon 174 is attached to the tapered nose cone 178, which is attached to a distal end portion of the inner catheter shaft 172. The valve stop member 190 is within the balloon 174 and longitudinally positioned between the outer catheter shaft 173 and the tapered nose cone 178. The valve stop member 190 is close to the tapered nose cone 178 than to the distal end of the outer catheter shaft 173.

FIG. 7 depicts yet another latter stage of the delivery/deployment process of the prosthetic heart valve 300 using the medical device delivery system 100. In this arrangement, the balloon 174 has been inflated and the prosthetic heart valve 300 has been radially expanded as a result. This expansion of the prosthetic heart valve 300 may be performed, for example, once the prosthetic heart valve 300 has been longitudinally and/or rotationally positioned properly in relation to a native heart valve annulus. That is, the unexpanded prosthetic heart valve 300 can be properly positioned in relation to the native anatomy, and then the prosthetic heart valve 300 can be expanded by inflation of the balloon 174 while remaining aligned with the native anatomy. Balloon catheter 170 is therefore part of an improved system for expanding prosthetic heart valve 300 while maintaining an orientation of prosthetic heart valve 300 relative to the native heart valve. This involves expanding prosthetic heart valve 300 while limiting a rotation of prosthetic heart valve 300 to less than a threshold amount of rotation. In some examples, this threshold amount of rotation is 10 degrees.

Because the balloon 174 is pleated, folded, and arranged to include folds that are deflected both clockwise and counterclockwise while the balloon 174 is disposed within the lumen 176 of the prosthetic heart valve 300 in the collapsed state, these folds apply pressure to the inner surface of prosthetic heart valve 300 as balloon 174 inflates in a way that causes the prosthetic heart valve 300 to expand radially outward while limiting a rotation of prosthetic heart valve 300 to less than a threshold amount of rotation. For example, the arrangement of balloon 174 ensures that rotational forces applied to prosthetic heart valve 300 by folds of the balloon 174 during expansion cancel to limit actual rotation of prosthetic heart valve 300. To the extent that the balloon 174 applies any net rotational force to prosthetic heart valve 300 as balloon 174 inflates, this rotation force is small enough such that the rotation of prosthetic heart valve 300 is less than a threshold amount of rotation. As a result, prosthetic heart valve 300 expands radially in response to the balloon 174 inflating.

In some embodiments, a rotation of prosthetic heart valve 300 relative to the inner catheter shaft 172 as a result of the balloon 174 inflating to the inflated state is less than a threshold amount of rotation. In some examples, this threshold amount of rotation is 10 degrees. For example, a difference in a rotational position of prosthetic heart valve 300 before balloon 174 is inflated while prosthetic heart valve 300 is in the collapsed state and a rotational position of prosthetic heart valve 300 after the prosthetic heart valve 300 is fully expanded can be less than the threshold amount of rotation (e.g., 10 degrees). The threshold amount of rotation is not limited to being 10 degrees. In some examples, the threshold amount of rotation can be any value within a range from zero degrees to 20 degrees (e.g., one degree, two degrees, five degrees, 15 degrees, or another value). In some examples, prosthetic heart valve 300 does not rotate at all in transitioning from the collapsed state to the expanded state.

Balloon 174 causes the prosthetic heart valve 300 to expand from a first diameter (D1) when the prosthetic heart valve 300 is in the collapsed state to a second diameter (D2) when the prosthetic heart valve 300 is in the expanded state. During this expansion, the inflation of the balloon 174 increases a diameter of the balloon, resulting in the diameter of the prosthetic heart valve 300 also increasing. As described above, when the balloon 174 is deflated, it is pleated to fit within a lumen 176 of the collapsed prosthetic heart valve 300. When the balloon 174 is fully inflated, the balloon 174 is no longer pleated and folded and has a round cross section. During expansion, the folds of the balloon membrane unfold so that the balloon can expand to its full diameter. When the prosthetic heart valve 300 is in the expanded state, the frame of prosthetic heart valve 300 can engage with the annulus of the native valve such that prosthetic heart valve 300 is secured at the targeted treatment site. This means that prosthetic heart valve 300 can operate as a healthy native valve does with blood flowing through the prosthetic heart valve leaflets in a forward direction and the leaflets blocking blood flow in the reverse direction.

Balloon catheter 170 and steerable catheter 160 can be withdrawn from the targeted treatment site when prosthetic heart valve 300 is in the expanded state and secured with the native valve annulus. In some cases, the balloon in the inflated state can be deflated via flush line 124 prior to withdrawing balloon catheter 170 and steerable catheter 160. When balloon catheter 170 and steerable catheter 160 are withdrawn, prosthetic heart valve 300 can remain in the expanded state and secured to the native valve annulus.

In the embodiment of FIG. 7, a proximal end of the balloon in the inflated state remains attached to attached to the outer catheter shaft 173 and a distal end of the balloon in the inflated state remains attached to tapered nose cone 178. In the inflated state, a diameter of the balloon 174 increases from the proximal end moving distally until the diameter reaches the greatest diameter D2. A midsection of the balloon has diameter D2. The diameter decreases from D2 to a smaller diameter at tapered nose cone 178 throughout a distal portion of the balloon 174. The collapsed prosthetic heart valve 300 is disposed on the middle portion of balloon 174 that achieves diameter D2. This means that a diameter of prosthetic heart valve 300 in the expanded state is greater than or equal to D2.

Referring now to FIGS. 5-7, prosthetic heart valve 300 includes a number of distinctive features that can serve as fiducial markers when prosthetic heart valve 300 is under visualization by medical imaging technology. In some embodiments, prosthetic heart valve 300 includes three commissure posts. For example, commissure post 312 and commissure post 313 are illustrated in FIG. 7. Prosthetic heart valve 300 also includes a third commissure post that is obstructed from view by balloon 174 in FIG. 7. These three commissure posts correspond to the commissures of the native aortic heart valve. In some embodiments, the commissure posts of prosthetic heart valve 300 are visible under visualization both when prosthetic heart valve 300 is in the collapsed state as illustrated in FIGS. 5-6 and when prosthetic heart valve 300 is in the expanded state as illustrated in FIG. 7. This means that a clinician operating the medical device delivery system 100 can align prosthetic heart valve 300 with the native aortic heart valve at the targeted site such that commissure posts of prosthetic heart valve 300 align with the commissures of the native aortic heart valve.

Aligning the commissure posts of prosthetic heart valve 300 with the commissures of the native heart valve can be achieved by rotating the balloon catheter 170 relative to steerable catheter 160 until the commissure posts of prosthetic heart valve 300 are aligned with the corresponding commissures of the native heart valve. Rotating balloon catheter 170 relative to steerable catheter 160 rotates prosthetic heart valve 300 in embodiments where prosthetic heart valve 300 is attached to rotating balloon catheter 170 when balloon catheter 170 is in the collapsed state. When the commissure posts of prosthetic heart valve 300 are aligned with the commissures of the native heart valve, the clinician can control the balloon 174 to inflate, thus causing prosthetic heart valve 300 to transition from the collapsed state to the expanded state. As the prosthetic heart valve 300 to transitions from the collapsed state to the expanded state, the commissure posts of the prosthetic heart valve 300 remain aligned with the commissures of the native heart valve because any rotation of prosthetic heart valve 300 about the delivery shaft of balloon catheter 170 during expansion does not exceed a threshold amount of rotation.

In some examples, for the prosthetic heart valve 300 to be “aligned” with the native heart valve, this involves a rotational position of the prosthetic heart valve 300 being less than a threshold amount of rotation displaced from a rotational position of the native heart valve. This threshold amount of rotation can be 10 degrees or another value. Displacement can occur in either direction (e.g., clockwise or counterclockwise), meaning that the range of acceptable rotational positions extends from a first rotational position in a first direction from the rotational position of the native valve to a second rotational position in a second direction from the rotational position of the native valve (e.g., acceptable range from 10 degrees clockwise of the native valve to 10 degrees counterclockwise of the native valve).

If the commissure posts of the prosthetic heart valve 300 are less than the threshold amount of rotation separated from the commissures of the native heart valve or the prosthetic heart valve leaflets of the prosthetic heart valve 300 are less than the threshold amount of rotation separated from the leaflets of the native valve, the prosthetic heart valve 300 is aligned with the native heart valve. In some embodiments, a clinician can control the prosthetic heart valve 300 to be aligned with the native heart valve within this range of acceptable rotational displacement, and the prosthetic heart valve 300 can remain within the range of acceptable rotational displacement as the prosthetic heart valve 300 transitions from the contracted state to the expanded state. Since balloon 174 expands radially outward due to the linear cavities defined by the pleating of the balloon 174, this limits rotational forces applied by the balloon 174 to prosthetic heart valve 300 during expansion.

Prosthetic heart valve 300 also includes a set of midpoint connectors that are spaced around a circumference of prosthetic heart valve 300 such that a midpoint connector is located midway between each pair of consecutive commissure posts. For example, midpoint connector 314 is located midway between commissure post 312 and commissure post 313. prosthetic heart valve 300 includes another two commissure posts that are obscured and not visible in FIG. 7. These midpoint connectors can serve as fiducial markers when balloon catheter 170 is under visualization during the delivery procedure. In some embodiments, each of the midpoint connectors defines a distinctive figure eight pattern that is visible in medical imaging such as a CT scan. This means that a clinician can identify the midpoint connectors relative to anatomical features of the patient. The midpoint connectors correspond to a midpoint of each prosthetic leaflet that is attached to the frame of the prosthetic heart valve 300. This means that clinicians can align prosthetic leaflets with native valve leaflets based on the location of the midpoint connectors in medical imaging. In some examples, the frame of prosthetic heart valve 300 is made of a fluoroscopic material such that the frame is visible under imaging from a CT scan an/or other imaging modalities.

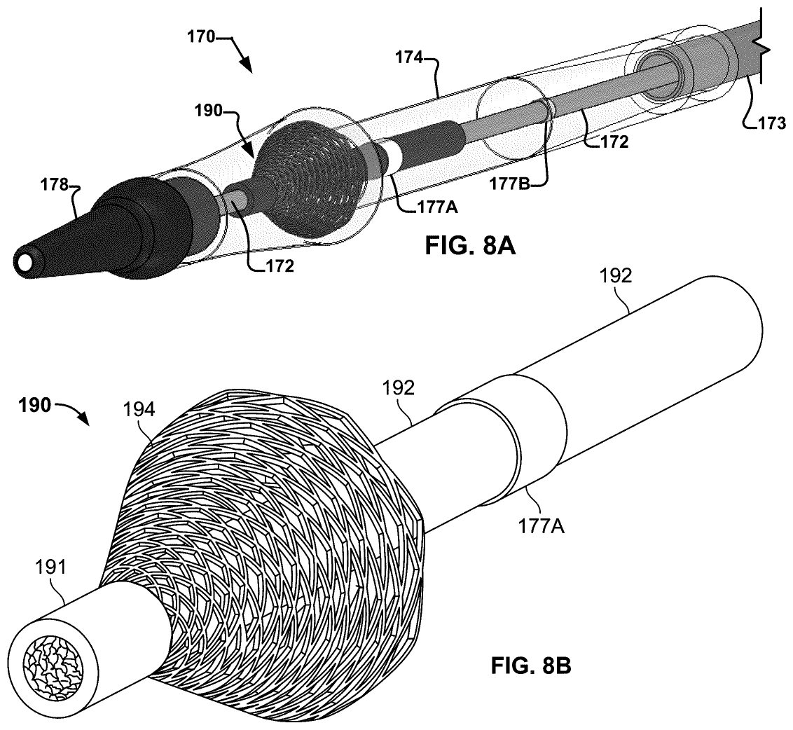

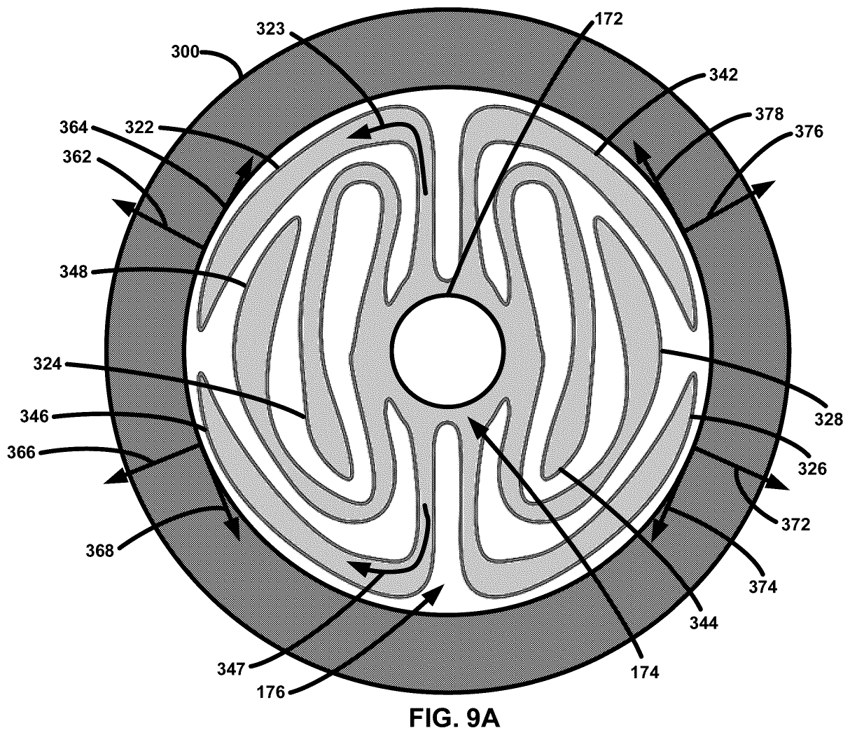

FIG. 8A illustrates a distal end portion of the balloon catheter 170. As shown, the balloon catheter 170 includes the inner catheter shaft 172, the outer catheter shaft 173, the balloon 174, the tapered nose cone 178, and the valve stop member 190. One or more radiopaque markers 177A-177B (collectively, “radiopaque markers 177”) can be located on various positions of the inner catheter shaft 172 and the valve stop member 190. In some embodiments, these one or more radiopaque markers are visible under visualization as the balloon catheter 170 advances through the vasculature of the patient towards the targeted site (e.g., the native aortic heart valve site). In the embodiment of FIG. 7, these radiopaque markers 177 are located on portions of the balloon catheter 170 that are proximate to the prosthetic heart valve 300 or within the lumen 176 of prosthetic heart valve 300 when the prosthetic heart valve 300 is in the collapsed state. This means that the clinician can determine a position of the prosthetic heart valve 300 relative to anatomy of the patient based on a position of the radiopaque markers 177 relative to the anatomy of the patient as the balloon catheter 170 advances.

Radiopaque marker 177A is located between a proximal end of prosthetic heart valve 300 and a distal end of prosthetic heart valve 300 when prosthetic heart valve 300 is disposed on balloon catheter 170. In some examples, radiopaque marker 177A is located near a center of prosthetic heart valve 300 when prosthetic heart valve 300 is in the expanded state, as illustrated in FIG. 7. In some examples, radiopaque marker 177B is located proximate to a proximal end of prosthetic heart valve 300 when prosthetic heart valve 300 is in the collapsed state, as illustrated in FIG. 6. This means that a clinician can determine a location of prosthetic heart valve 300 based on a position of radiopaque markers 177 relative to anatomy of the patient. Radiopaque markers 177 are not required for determining the position of balloon catheter 170. In some cases, a clinician can determine the position of balloon catheter 170 based on one or more fiducial markers located on the prosthetic heart valve 300 itself such as commissure posts 312, 313 and midpoint connector 314.

FIG. 8B shows the valve stop member 190 in isolation so that additional details of its construction are visible. The valve stop member 190 includes a distal hub 191, an elongate proximal hub 192, and a frustoconical surface 194. Valve stop member 190 also includes a radiopaque marker 177A located on a surface of valve stop member 190. This radiopaque marker 177A is located proximal to the frustoconical surface 194.

The distal hub 191 is attached/affixed to the inner catheter shaft 172 such that it is held in a constant position. The elongate proximal hub 192, however, is a polymeric or metallic tube that is slidable along the inner catheter shaft 172 rather than being attached to the inner catheter shaft 172. More particularly, the elongate proximal hub 192 comprises an elongate tube that defines a lumen in which the inner catheter shaft 172 is slidably disposed. The elongate proximal hub 192 can slide along the inner catheter shaft 172. As described further below, during the assembly of the balloon catheter 170 the elongate proximal hub 192 is forcibly slid along the inner catheter shaft 172 to longitudinally stretch the valve stop member 190 and to thereby reduce the outer diameter of the frustoconical surface 194 so that the balloon 174 can be moved into position over the valve stop member 190.

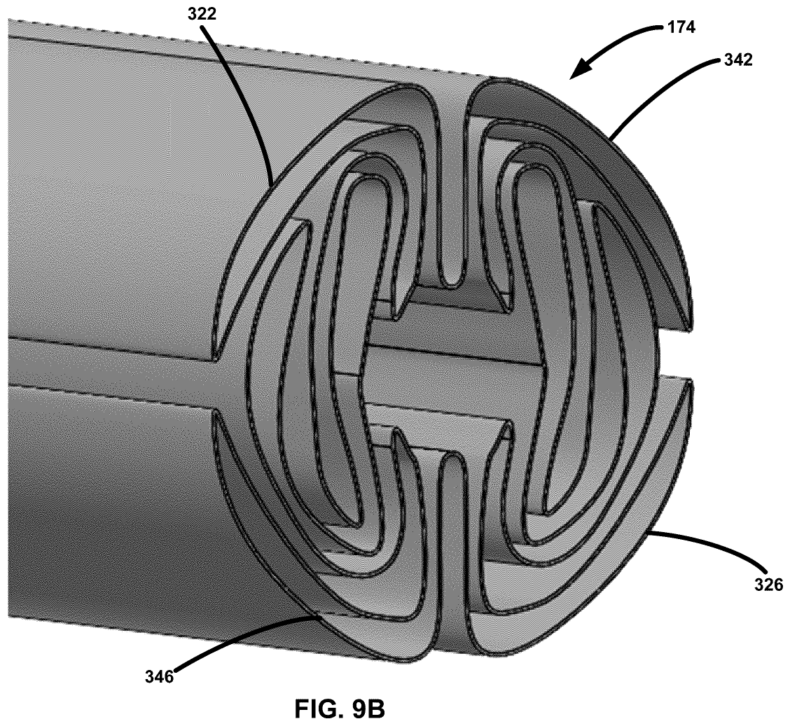

FIG. 9A is a diagram illustrating an enlarged version of the cutaway view of the balloon catheter 170 at position X illustrated in FIG. 4, the enlarged version indicating one or more forces applied by the balloon 174 to the prosthetic heart valve 300. As seen in FIG. 9A, balloon 174 can be pleated, folded, and arranged to fit within lumen 176 when balloon 174 is in the deflated state. For example, balloon 174 includes the first set of folds 322-328 and the second set of folds 342-348. Each of the first set of folds 322-328 is deflected in a first rotational direction (e.g., counterclockwise) relative to a longitudinal axis of the balloon catheter 170 that extends into and out of the page from the perspective of the cutaway view of FIG. 9A. Each of the second set of folds 342-348 is deflected in a second rotational direction (e.g., clockwise) relative to a longitudinal axis. For example, fold 322 is deflected counterclockwise according to deflection 323 and fold 346 is deflected clockwise according to deflection 347.

Since fold 322 and fold 346 are deflected or wrapped to extend in opposite directions (i.e., fold 322 deflected counterclockwise and fold 346 deflected clockwise) so that deflated balloon 174 can fit within lumen 176 of the collapsed prosthetic heart valve 300, fold 322 and fold 346 apply opposing rotational forces to the inner surface of prosthetic heart valve 300 as prosthetic heart valve 300 transitions from the collapsed state to the expanded state. As seen in FIG. 9A, fold 322 can apply a first force to prosthetic heart valve 300 that has a radial component 362 and a tangential component 364 and fold 346 can apply a second force to prosthetic heart valve 300 that has a radial component 366 and a tangential component 368. The tangential components 364, 368 of these forces represent rotational forces that alone and unopposed would cause prosthetic heart valve 300 to rotate. However, as seen in FIG. 9A, tangential component 364 of the first force applied by fold 322 and tangential component 368 of the second force applied by fold 346 are applied in opposite directions, meaning that these components substantially cancel each other. Radial component 362 and radial component 366 are both directed outwards, meaning that radial component 362 and radial component 366 work together to cause prosthetic heart valve 300 to expand radially outward. The net result of the collective radial force components and cancelling tangential force components is that prosthetic heart valve 300 expands radially outward without rotating substantially about the longitudinal axis of the balloon catheter 170.

Fold 322 and fold 346 thus represent an opposing pair of deflected folds. The example arrangement of the balloon 174 illustrated in FIG. 9A includes three other opposing pairs of deflected folds that each work together to combine radial forces and cancel tangential forces applied to prosthetic heart valve 300. Fold 326 and fold 342 and represent another such pair of opposing deflected folds, with fold 326 deflected in the counterclockwise direction and fold 342 deflected in the clockwise direction. This results in fold 326 applying a third force including a radial component 372 and a tangential component 374 and fold 328 applying a fourth force including a radial component 376 and a tangential component 378. As with the pair of fold 322 and fold 346, the pair including fold 326 and fold 342 apply opposing tangential forces to prosthetic heart valve 300 (i.e., tangential component 374 and tangential component 378 are in opposite directions) and complimentary radial forces (i.e., radial component 372 and radial component 376 are both directed outward). This means that folds 326, 342, much like folds 322, 346, represent a pair of opposing deflected folds that promote radial expansion of prosthetic heart valve 300 while limiting rotation of prosthetic heart valve 300 about longitudinal axis. Folds 324, 348 and folds 328, 344 are two more pairs of opposing deflected folds that also promote radial expansion while limiting rotation.

In aggregate, the forces applied by the first set of folds 322-328 and the second set of folds 342-348 as balloon 174 expands from the deflated state to the inflated state include a net outward radial force that causes prosthetic heart valve 300 to increase in diameter. To the extent that the first set of folds 322-328 and the second set of folds 342-348 apply a net rotational force, to prosthetic heart valve 300, this force is limited such that a rotation of prosthetic heart valve 300 during expansion is limited to less than a threshold amount of rotation (e.g., less than 10 degrees). Since the first set of folds 322-328 are deflected in a first rotational direction and the second set of folds 342-348 are deflected in a second rotational direction, this means that the collective tangential forces applied by the first set of folds 322-328 is substantially equal to the collective tangential forces applied by the second set of folds 342-348 during expansion. Even in cases where the tangential forces applied by the first set of folds 322-328 and the tangential forces applied by the second set of folds 342-348 do not perfectly cancel, the net tangential force may be small enough so that any resulting rotation of prosthetic heart valve 300 is less than the threshold amount of rotation.

FIG. 9B is a diagram illustrating a cutaway cross-sectional perspective view of the balloon 174 arranged according to the pattern illustrated in FIG. 9A to fit within a lumen of the collapsed prosthetic heart valve 300. As seen in FIG. 9B, balloon 174 is arranged to deflect a plurality of folds so that balloon 174 fits within a diameter that is significantly smaller than a diameter of the balloon 174 when fully expanded. Balloon 174 extends along a longitudinal axis of balloon catheter 170, with the folds extending along the longitudinal axis of balloon catheter 170. This means that the cutaway view illustrated in FIG. 9A represents the pattern of the balloon 174 that is present in cutaways along a significant portion of the balloon 174. The opposing pairs of folds 322, 346 and folds 326, 242 are shown in FIG. 9B. Other pairs of folds are also present in the example of FIG. 9B.

This disclosure is not limited to the pattern illustrated in FIGS. 4 and 9A-9B. The balloon 174 when deflated can form one or more other patterns that cause prosthetic heart valve 300 to expand while a rotation of prosthetic heart valve 300 is limited. For example, there may be one or more other patterns that include folds that apply opposing tangential forces to the prosthetic heart valve 300 during expansion to limit rotation of prosthetic heart valve 300.

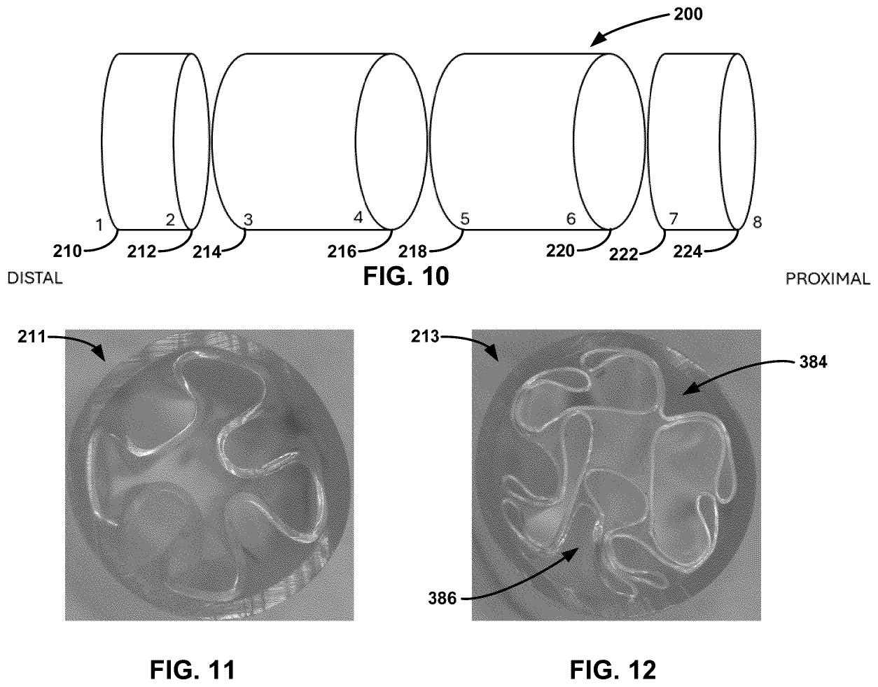

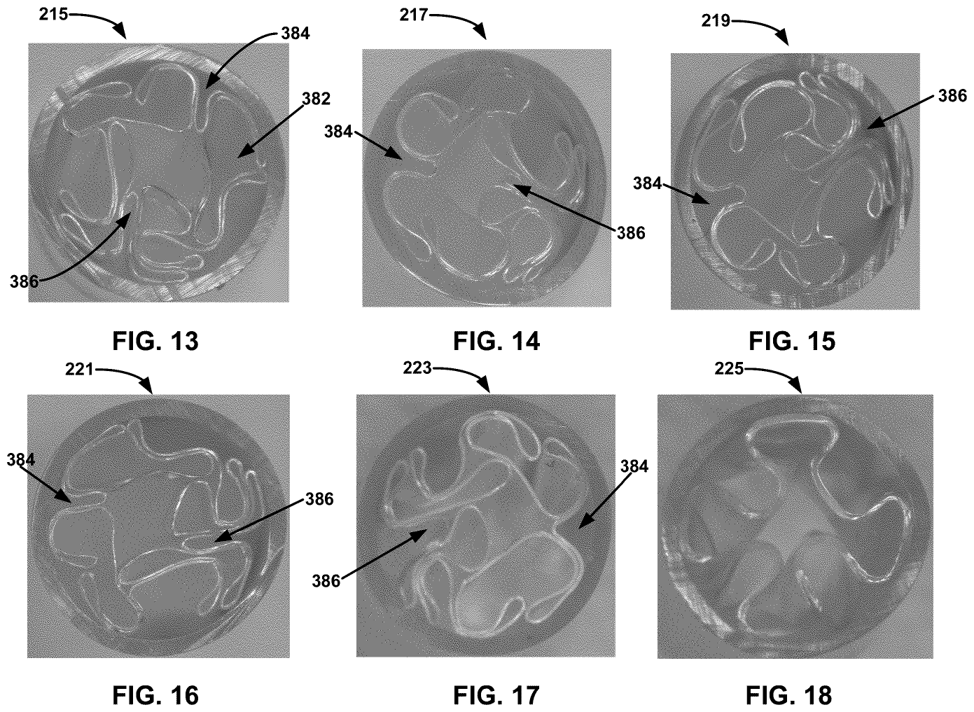

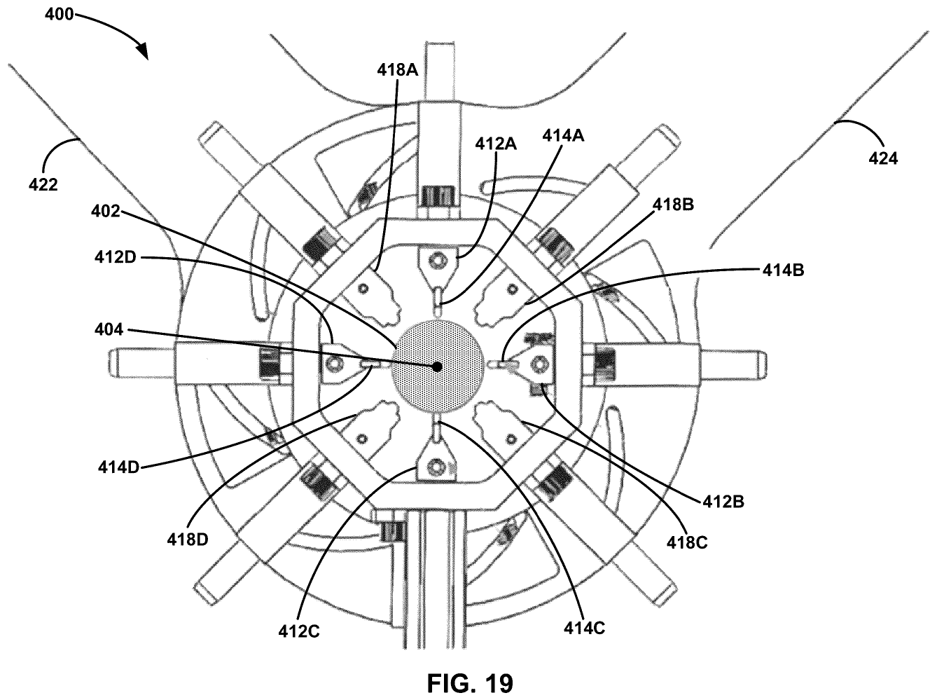

FIG. 10 is a diagram of a set of cutaway locations 210-224 of balloon 174 in the deflated state while disposed within the lumen of prosthetic heart valve 300 in the compressed state. Cutaway location 210 is a distalmost cutaway location, cutaway location 224 is the furthest proximal cutaway location, and cutaway locations 212-222 are located between cutaway location 210 and cutaway location 224. Each cutaway location corresponds to a respective cross-section illustrated in FIGS. 10-18, as described in further detail below. For example, cutaway location 210 corresponds to FIG. 11, cutaway location 212 corresponds to FIG. 12, cutaway location 214 corresponds to FIG. 13, cutaway location 216 corresponds to FIG. 14, cutaway location 218 corresponds to FIG. 15, cutaway location 220 corresponds to FIG. 16, cutaway location 222 corresponds to FIG. 17, and cutaway location 224 corresponds to FIG. 18.

FIGS. 11-18 illustrate cutaway cross-sectional views of balloon 174 in the deflated state while disposed within the lumen of prosthetic heart valve 300 in the compressed state at each of the cutaway locations 210-224 of FIG. 10. The arrangement of balloon 174 in the embodiment illustrated in FIGS. 11-18 is different than the arrangement of balloon 174 in the embodiment illustrated in FIGS. 4 and 9A-9B. For example, FIG. 11 illustrates a cutaway view 211 of balloon 174 corresponding to cutaway location 210, FIG. 12 illustrates a cutaway view 213 corresponding to cutaway location 212, FIG. 13 illustrates a cutaway view 215 corresponding to cutaway location 214, FIG. 14 illustrates a cutaway view 217 corresponding to cutaway location 216, FIG. 15 illustrates a cutaway view 219 corresponding to cutaway location 218, FIG. 16 illustrates a cutaway view 221 corresponding to cutaway location 220, FIG. 17 illustrates a cutaway view 223 corresponding to cutaway location 222, and FIG. 18 illustrates a cutaway view 225 corresponding to cutaway location 224.

The cutaway view 211 of FIG. 11, for example, shows that balloon 174 forms a cloverleaf pattern at the distalmost cutaway location 210. Balloon 174 does not form any folds in the cutaway view 211. As illustrated in FIG. 7, a maximum diameter of balloon 174 at the distal end of the balloon is less than the maximum diameter of balloon 174 in a middle section of balloon 174. This means that the balloon 174 does not have as much membrane material to fit within the lumen of prosthetic heart valve 300 at cutaway location 210 as the amount of membrane material that balloon 174 fits within the lumen of prosthetic heart valve 300 at cutaway views in the middle section of balloon 174. The same is true for the cutaway view 225 of FIG. 18, which corresponds to the most proximal cutaway location 224 of FIG. 10. In cutaway view 225, balloon 174 forms a cloverleaf pattern that is very similar to the cloverleaf pattern of balloon 174 in the cutaway view 211 corresponding to the distalmost cutaway location 210.

Cutaway views 213-223 correspond to cutaway locations 212-222 that are located between the most distal cutaway location 210 and the most proximal cutaway location 224. These cutaway views 213-223 show that balloon 174 is pleated and folded so that the membrane of balloon 174 forms several folds that define recesses that extend for a substantial portion of the length of balloon 174. For example, open recess 384 is visible in each of cutaway views 213-223 and is defined by folds of balloon 174 that bend away from each other. Open recess 384 is substantially linear and extends parallel relative to inner catheter shaft 172 and a longitudinal axis of balloon 174. Since open recess 384 is linear, open recess 384 extends parallel to inner catheter shaft 172. Additionally, open recess 386 is formed by two folds that bend outwards away from each other, forming an open channel. Open recess 386 is substantially linear and extends parallel relative to inner catheter shaft 172 and a longitudinal axis of balloon 174. Since open recess 386 is linear, open recess 386 extends parallel to inner catheter shaft 172.

Cutaway views 213-223 show several other open recesses and closed recesses. Each of these recesses is linear and extends parallel to inner catheter shaft 172 and a longitudinal axis of balloon 174. This means that when balloon 174 is pleated and folded to fit within the lumen of prosthetic heart valve 300 in the compressed state, balloon 174 is pleated and folded to form linear recesses that follow a point on the circumference of inner catheter shaft 172. For example, open recess 384 forms a valley, with a bottom of the valley extending in a substantially straight line. Since open recess 384 is linear, this valley expands radially outwards in a direction that is substantially perpendicular to a tangent of balloon 174 and without expanding rotationally. Open recess 386 forms an open channel. During expansion, the folds that define open recess 386 extend outward and the bottom of the closed channel expands radially in a direction that is substantially perpendicular to a tangent of balloon 174 and without expanding rotationally.

FIG. 19 illustrates a pleating tool 400 for pleating a balloon 402 to fit within the recess of a prosthetic heart valve in the collapsed state. Pleating tool 400 is configured to pleat balloon 402 so that the membrane of balloon 402 forms linear recesses that extend along a longitudinal axis of the balloon. As seen in FIG. 19, pleating tool 400 includes a mandrel 404. Pleating tool 400 also includes pleating heads 412A-412D (collectively, “pleating heads 412”). Each pleating head of pleating heads 412 corresponds to a pleating tooth of pleating teeth 414A-414D (collectively, “pleating teeth 414”). Pleating tool 400 also includes folding heads 418A-418D (collectively, “folding heads 418”).

As seen in FIG. 19, pleating heads 412, pleating teeth 414, and folding heads 418 are each aligned to extend radially along an axis that intersects with a center axis of pleating tool 400. Mandrel 404 extends along the center axis of pleating tool 400. In some examples, pleating heads 412, pleating teeth 414, and folding heads 418 are configured to move inwards and outwards radially toward mandrel 404 and away from mandrel 404. For example, pleating heads 412 and pleating teeth 414 are configured to move radially inwards in response to a clockwise rotation of arm 422 and move radially outwards in response to a counterclockwise rotation of arm 422. Folding heads 418 are configured to move radially inwards in response to a clockwise rotation of arm 424 and move radially outwards in response to a counterclockwise rotation of arm 424.

Pleating teeth 414 are removably attached to pleating heads 412. For example, pleating tooth 414A is removably attached to pleating head 412A, pleating tooth 414B is removably attached to pleating head 412B, pleating tooth 414C is removably attached to pleating head 412C, and pleating tooth 414D is removably attached to pleating head 412D. Balloon 402 is mounted on the mandrel 404 in a center of pleating tool 400. As seen in FIG. 19, a cross-section of balloon 402 is round, as the balloon is in an inflated state or a semi-inflated state and has not yet been pleated. As pleating teeth 414 and pleating heads 412 and/or folding heads 418 move inwards, this will cause the membrane of balloon 402 to deform such that the cross section is no longer round. When the balloon 402 is round and no pleating has yet been performed, this is the “first pleating position” of pleating tool 400.

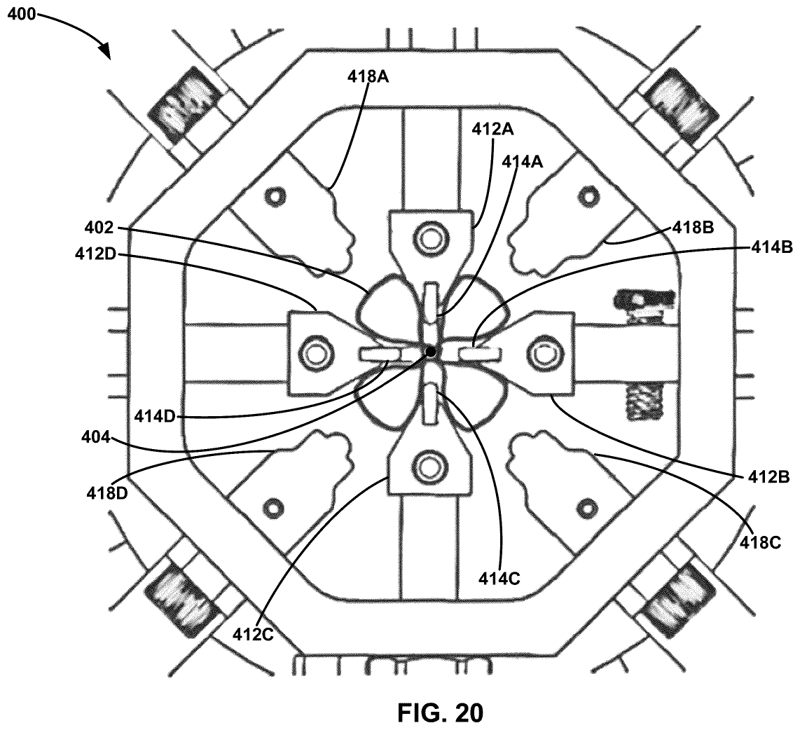

FIG. 20 illustrates pleating tool 400 for pleating a balloon 402 in a second pleating position where pleating heads 412 and pleating teeth 414 are extended inwards to create a set of cavities. For example, pleating head 412A and pleating tooth 414A can extend radially inward to create a first cavity on the top side of balloon 402, pleating head 412B and pleating tooth 414B can extend radially inward to create a second cavity on the right side of balloon 402, pleating head 412C and pleating tooth 414C can extend radially inward to create a third cavity on the bottom side of balloon 402, and pleating head 412D and pleating tooth 414D can extend radially inward to create a first cavity on the left side of balloon 402. As seen in FIG. 20, a cross section of balloon 402 forms a cloverleaf pattern in the second pleating position of pleating tool 400, the cloverleaf pattern including four bulges of the membrane of balloon 402. Each bulge of the four bulges is between two consecutive pleating heads of pleating heads 412. Pleating tool 400 can transition to the second pleating position of FIG. 12 based on a clockwise rotation of arm 422 of FIG. 19, which advances pleating heads 412 and pleating theeth 414 inwards.

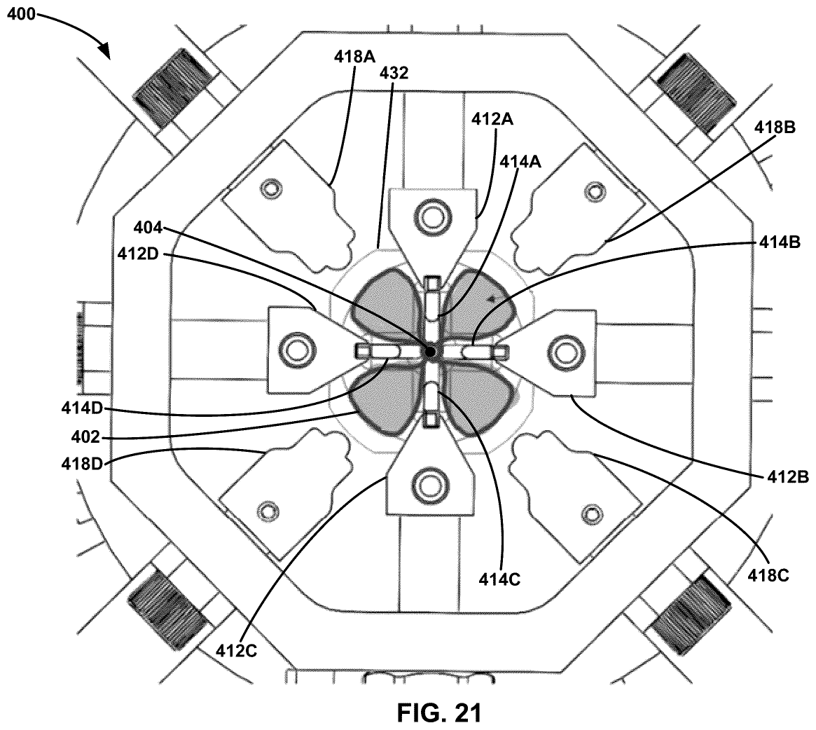

FIG. 21 illustrates pleating tool 400 for pleating a balloon 402 in a third pleating position where pleating heads 412 are retreated and pleating teeth 414 preserve cavities in the balloon 402 and a pleating nut 432 is attached to secure pleating teeth 414 in place. For example, pleating nut 432 can fit over pleating teeth 414 such that pleating teeth 414 are secured to pleating nut 432 and pleating teeth 414 are free to disengage from pleating heads 412. For example, pleating nut 432 can secure pleating teeth 414 in place so that the cloverleaf pattern and the cavities of balloon 402 is preserved even if pleating heads 412 disengage and retreat from pleating teeth 414. This means that pleating nut 432 can secure pleating teeth 414 in place without securing pleating heads 412 in place.

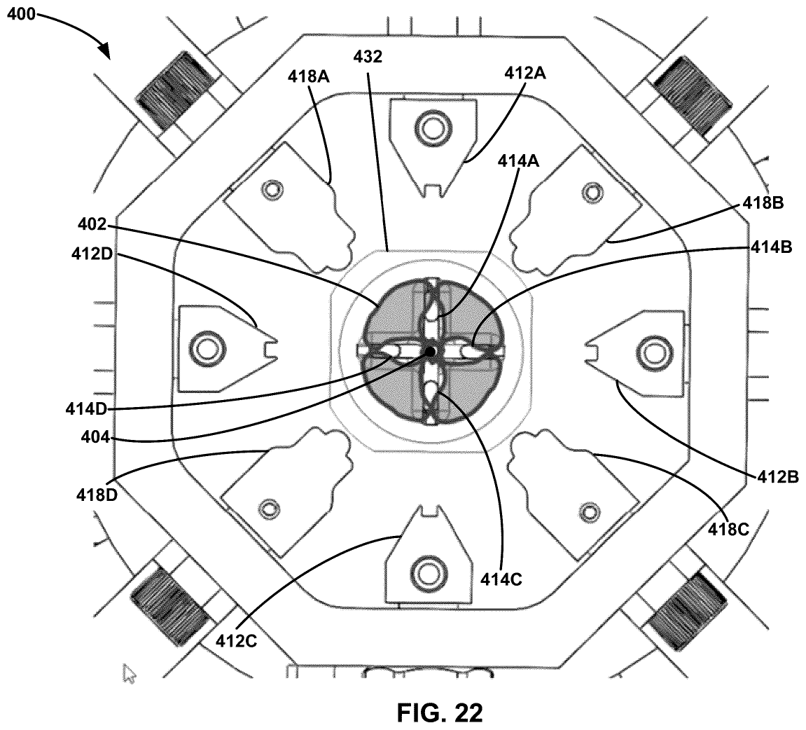

FIG. 22 illustrates pleating tool 400 for pleating a balloon 402 in a fourth pleating position where pleating heads 412 are retreated and pleating teeth 414 preserve cavities in the balloon 402 while secured by pleating nut 432. In some examples, following the second pleating position illustrated in FIG. 20 where pleating heads 412 and pleating teeth 414 advance to create cavities in balloon 402, pleating nut 432 can be placed on pleating tool 400 to secure pleating teeth 414 in place When pleating nut 432 secures pleating teeth 414, pleating heads 412 can radially retract in response to a counterclockwise rotation of arm 422, leaving pleating teeth 414 in place to secure the cavities in balloon 402. As seen in FIG. 21, balloon 402 maintains a cloverleaf pattern secured by pleating nut 432 and pleating teeth 414 when the pleating heads 412 retract. As seen in FIG. 21, in the second pleating position the pleating heads 412 are separated from the pleating teeth. In some examples, pleating heads 412 retreat so that folding heads 418 can advance inwards without being obstructed by pleating heads 412.

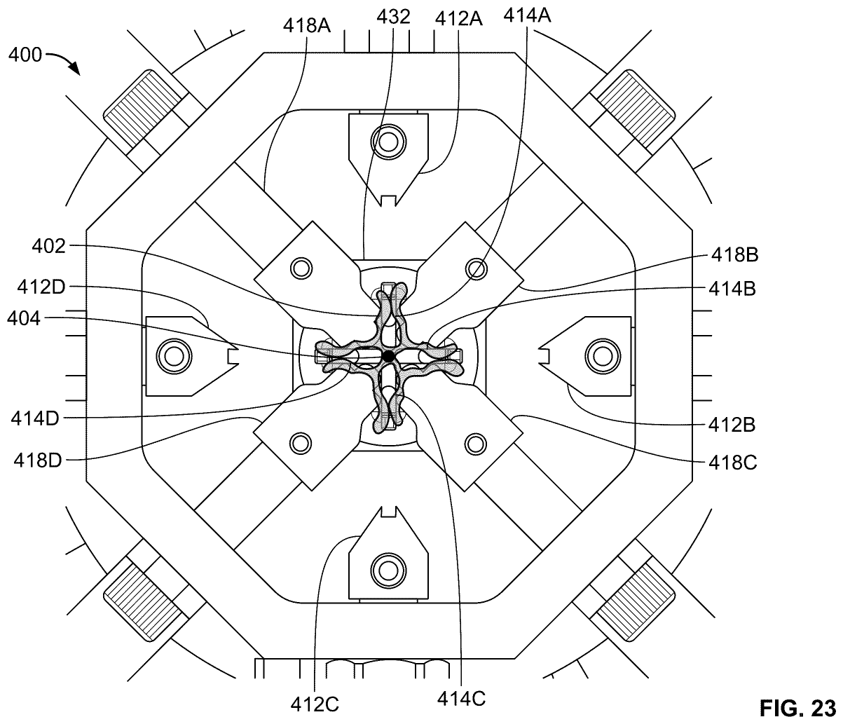

FIG. 23 illustrates pleating tool 400 for pleating a balloon 402 in a fifth pleating position where folding heads 418 are advanced to create folds in the membrane of the balloon 402. For example, folding heads 418 can advance in response to a counterclockwise rotation of arm 424 illustrated in FIG. 19. Folding heads 418 can advance inward radially, compressing the bulges of the cloverleaf pattern of the balloon 402 that are present in the fourth pleating position. As seen in FIG. 23, when the folding heads 418 are advanced, this causes the membrane of the balloon to fold over itself in several locations, referred to herein as “folds” of the balloon 402. These folds can be further manipulated to decrease a radius of the balloon 402 in the deflated state. The folding heads 418 create recesses in the balloon membrane. These recesses are linear, meaning that the recesses extend along the balloon substantially parallel to a longitudinal axis of the balloon.

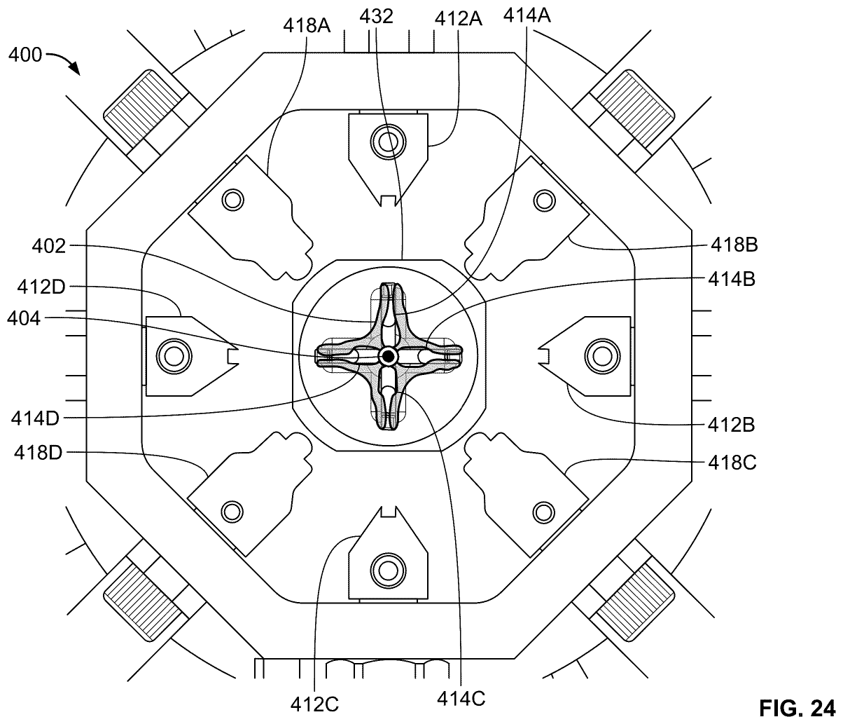

FIG. 24 illustrates pleating tool 400 for pleating a balloon 402 in a sixth pleating position where pleating heads 412 and folding heads 418 are retracted and the folds in the membrane of the balloon 402 are preserved by vacuum. For example, pleating tool 400 can vacuum air and/or liquid from the balloon 402 so that the shape of balloon 402 is maintained. This allows folding heads 418 to retreat with balloon 402 remaining in the same shape with folds. In some examples, folding heads 418 retract in response to a clockwise movement of arm 424 illustrated in FIG. 19. When folding heads 418 are retracted, the pleating nut 434 can be removed. At this point, the folds of the balloon 174 can be further arranged to form open cavities and closed cavities such that balloon 174 fits within a lumen of prosthetic heart valve 300 while in the collapsed position. That is, equal numbers of the folded portions of the balloon 402 can be wrapped to extend around the longitudinal axis of the balloon 402 in opposing directions (e.g., a first four folded portions can be wrapped clockwise and the remaining four folded portions can be wrapped counterclockwise).

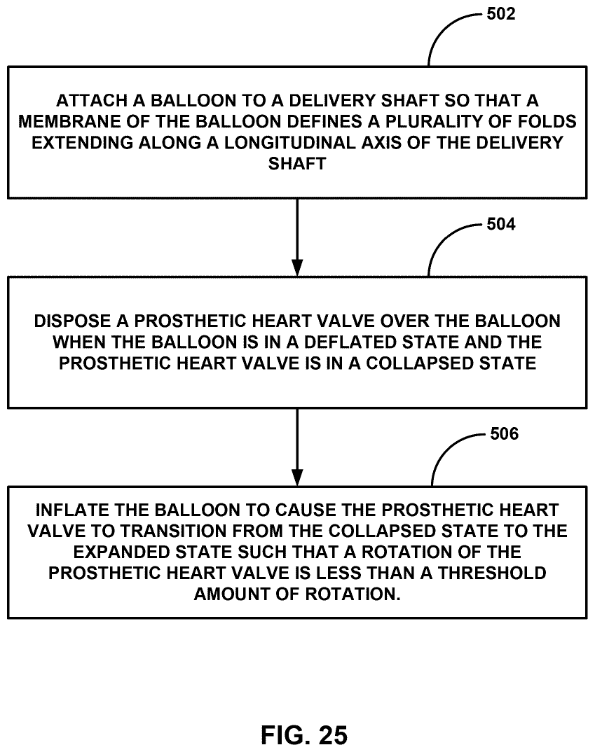

FIG. 25 is a flow diagram illustrating an example operation for expanding a balloon catheter including a pleated balloon with linear recesses. FIG. 25 is described with respect to medical device delivery system 100 of FIGS. 1-7. However, the techniques of FIG. 25 may be performed by different components of medical device delivery system 100 or by additional or alternative medical device systems.

A balloon 127 can be attached to a delivery shaft so that a membrane of balloon 127 defines a plurality of folds extending along a longitudinal axis of the delivery shaft (502). In some cases, the balloon 174 can be attached to any part of the delivery shaft of balloon catheter 170 including tapered nose cone 178, inner catheter shaft 172, outer catheter shaft 173, valve stop member 190, or any combination thereof. In some examples, a distal end of balloon 174 is attached to tapered nose cone 178 and a proximal end of balloon 174 is attached to outer catheter shaft 173. Outer catheter shaft 173 can be slidably received by a lumen of a steerable catheter 160 which is proximal to the balloon catheter 170. This means that balloon catheter 170 is configured to slide distally away from steerable catheter 160 or move distally towards steerable catheter 160. In some examples, the plurality of folds of balloon 174 can include a first set of folds deflected in a first direction and a second set of folds deflected in a second direction. In some examples, the first set of folds includes four folds and the second set of folds includes four folds.

The plurality of folds occur when a flap of membrane material folds over itself so that the membrane material is two layers thick with a transition portion where the material folds over. These folds can be arranged to occupy a small footprint so that the balloon 174 can fit within a lumen that has a diameter significantly smaller than a diameter of balloon 174 when inflated. For example, the folds of the balloon 174 in the deflated state can be arranged to form one or more open recesses of the plurality of recesses and one or more closed recesses of the plurality of recesses. Each closed recess of the one or more closed recesses can be defined by overlapping folds of the membrane of balloon 174. Each open recess of the one or more open recesses can formed by folds of the membrane of balloon 174 extending away from each other to create a valley. Open recess and closed recesses both extend parallel to the delivery shaft and parallel to a longitudinal axis of balloon catheter 170.

In some embodiments, a prosthetic heart valve 300 is disposed over the balloon 174 that is fixedly attached to the delivery shaft of the balloon catheter 170 (504). Prosthetic heart valve 300 can be in the collapsed state while balloon 174 in the deflated state is disposed within a lumen defined by the collapsed prosthetic heart valve 300. Since balloon 174 can be pleated in the deflated state to occupy a cross-section having a diameter significantly smaller than the balloon 174 when inflated, the balloon 174 can fit within the lumen defined by prosthetic heart valve 300 in the collapsed state. Prosthetic heart valve 300 can be secured in place by valve stop member 190 and a flared distal end 162 of the steerable catheter 160. This allows balloon catheter 170 to be advanced to the targeted treatment side (e.g., the native aortic heart valve) with the prosthetic heart valve 300 secured to the balloon catheter 170, the prosthetic heart valve 300 in the collapsed state and the balloon 174 in the deflated state.

Balloon 187 can inflate, causing the prosthetic heart valve 300 to transition from the collapsed state to the expanded state such that a rotation of the prosthetic heart valve 300 about a longitudinal axis of the delivery shaft is less than a threshold amount of rotation (508). This allows a clinician to align prosthetic heart valve 300 with the native valve while prosthetic heart valve 300 is collapsed such that the prosthetic heart valve 300 remains aligned with the prosthetic heart valve 300 when expanded. In some examples, the folds deflected in opposite directions promote radial expansion of prosthetic heart valve 300 without rotation since tangential forces applied by folds deflected in opposite directions cancel each other out.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. A balloon catheter system for delivering a prosthetic heart valve to a targeted site at a native valve of a patient, the balloon catheter system comprising:
a delivery shaft;
a balloon fixedly attached to the delivery shaft, wherein when the balloon is in a deflated state, a membrane material of the balloon defines a plurality of folded portions extending along a longitudinal axis of the delivery shaft; and
a prosthetic heart valve disposed over the balloon when the balloon is in the deflated state and the prosthetic heart valve is in a collapsed state, a first set of folded portions of the plurality of folded portions extend clockwise about the longitudinal axis of the delivery shaft and a second set of folded portions of the plurality of folded portions extend counterclockwise about the longitudinal axis of the delivery shaft.

2. The balloon catheter of claim 1, wherein the balloon is configured such that, as the balloon transitions from the deflated state to an inflated state, the first set of folded portions expand outwards relative to the longitudinal axis and counterclockwise about the longitudinal axis and the second set of folded portions expand outwards relative to the longitudinal axis and clockwise about the longitudinal axis to cause the prosthetic heart valve to transition from the collapsed state to an expanded state.

3. The balloon catheter of claim 2, wherein as the first set of folded portions expand counterclockwise about the longitudinal axis and the second set of folded portions expand clockwise about the longitudinal axis, the first set of folded portions apply a first rotational force to the prosthetic heart valve and the second set of folded portions apply a second rotational force to the prosthetic heart valve that is opposite the first rotational force

4. The balloon catheter system of claim 2, wherein a rotation of the prosthetic heart valve relative to the delivery shaft in response to the prosthetic heart valve transitioning from the collapsed state to the expanded state is less than a threshold amount of rotation.

5. The balloon catheter system of claim 4, wherein the threshold amount of rotation is 10 degrees.

6. The balloon catheter system of claim 2,
wherein the delivery shaft is configured to be advanced to the targeted site when the balloon is in the deflated state and the prosthetic heart valve is in the collapsed state so that each prosthetic leaflet of a set of prosthetic leaflets attached to the prosthetic heart valve is aligned with a corresponding native leaflet of the native valve, and
wherein in response to the balloon transitioning from the deflated state to an inflated state, the prosthetic heart valve expands from the collapsed state to the expanded state with each prosthetic leaflet of the set of prosthetic leaflets remaining aligned with the corresponding native leaflet of the native valve when the prosthetic heart valve is in the expanded state.

7. The balloon catheter system of claim 6, wherein the delivery shaft and the balloon are configured to be withdrawn from the targeted site, leaving the prosthetic heart valve in the expanded state with each prosthetic heart valve leaflet of the set of prosthetic heart valve leaflets remaining aligned with the corresponding native leaflet of the native valve.

8. The balloon catheter system of claim 1, wherein the first set of folded portions and the second set of folded portions define a plurality of recesses wherein each recess of the plurality of recesses extends along the longitudinal axis of the delivery shaft.

9. The balloon catheter system of claim 8, wherein the plurality of recesses comprise:
one or more enclosed recesses, each enclosed recess of the one or more enclosed recesses defined by a folded portion of the first set of folded portions and a folded portion of the second set of folded portions deflected towards each other; and
one or more open recesses, each open recess of the one or more open recesses defined by a folded portion of the first set of folded portions and a folded portion of the second set of folded portions deflected away from each other.

10. The balloon catheter system of claim 1, wherein the plurality of folded portions comprise one or more pairs of folded portions, each pair of folded portions of the one or more pairs of folded portions comprising:
a folded portion of the first set of folded portions extending clockwise about the longitudinal axis of the delivery shaft; and
a folded portion of the second set of folded portions extending counterclockwise about the longitudinal axis of the delivery shaft.

11. The balloon catheter system of claim 1, wherein the plurality of folded portions comprises eight folded portions, wherein the first set of folded portions comprises four folded portions, and wherein the second set of folded portions comprises four folded portions.

12. The balloon catheter system of claim 1:
wherein the delivery shaft is configured to be advanced to the targeted site when the balloon is in the deflated state and the prosthetic heart valve is in the collapsed state, wherein the prosthetic heart valve in the collapsed state is configured to rotate about the longitudinal axis of the delivery shaft, and
wherein based on the prosthetic heart valve in the collapsed state being within a desired range of rotational positions relative to the native valve of the patient, the balloon is configured to inflate to cause the prosthetic heart valve to transition from the collapsed state to an expanded state, the prosthetic heart valve in the expanded state remaining within the desired range of rotational positions relative to the native valve of the patient.

13. The balloon catheter system of claim 12, wherein the desired range of rotational positions relative to the native valve of the patient comprises a target rotational position, and wherein the desired range of rotational positions extends from a first rotational position displaced counterclockwise from the target rotational position to a second rotational position displaced clockwise from the target rotational position.

14. The balloon catheter system of claim 13, wherein the first rotational position is displaced 10 degrees counterclockwise from the target rotational position and the second rotational position is displaced 10 degrees clockwise from the target rotational position.

15. The balloon catheter system of claim 1, wherein the prosthetic heart valve comprises:
a frame configured to expand from the collapsed state to the expanded state in response to the balloon transitioning from the deflated state to an inflated state; and
the set of prosthetic heart valve leaflets attached to the expandable frame, wherein as the membrane material of the balloon expands radially outward relative to the delivery shaft, the balloon applies a radial force to an inside surface of the frame such that the frame expands radially outward relative to the delivery shaft from the collapsed state to the expanded state.

16. A balloon catheter for delivering a prosthetic heart valve to a targeted site at a native valve of a patient, the balloon catheter comprising:
a delivery shaft; and
a balloon fixedly attached to the delivery shaft, wherein when the balloon is in a deflated state, a membrane material of the balloon defines a plurality of folded portions extending along a longitudinal axis of the delivery shaft,
wherein when the balloon is disposed within a prosthetic heart valve when the balloon is in the deflated state and the prosthetic heart valve is in a collapsed state, a first set of folded portions of the plurality of folded portions extend clockwise about the longitudinal axis of the delivery shaft and a second set of folded portions of the plurality of folded portions extend counterclockwise about the longitudinal axis of the delivery shaft.

17. The balloon catheter of claim 16, wherein the first set of folded portions and the second set of folded portions define a plurality of recesses wherein each recess of the plurality of recesses extends along the longitudinal axis of the delivery shaft.

18. The balloon catheter of claim 17, wherein the plurality of recesses comprise:
one or more enclosed recesses, each enclosed recess of the one or more enclosed recesses defined by a folded portion of the first set of folded portions and a folded portion of the second set of folded portions deflected towards each other; and
one or more open recesses, each open recess of the one or more open recesses defined by a folded portion of the first set of folded portions and a folded portion of the second set of folded portions deflected away from each other.

19. The balloon catheter of claim 18, wherein the plurality of folded portions comprise one or more pairs of folded portions, each pair of folded portions of the one or more pairs of folded portions comprising:
a folded portion of the first set of folded portions deflected clockwise about the longitudinal axis of the delivery shaft; and
a folded portion of the second set of folded portions deflected counterclockwise about the longitudinal axis of the delivery shaft.

20. A method of pleating a balloon attached to a delivery shaft comprises:
inflating the balloon such that a cross-section of the balloon is substantially circular;
moving a set of pleating heads radially inward, each pleating head of the set of pleating heads pushing a membrane material of the balloon inwards to define a recess of a set of recesses, wherein each pleating head comprises a pleating tooth located at a distal end of the respective pleating head;
placing a pleating nut to secure the pleating tooth of each pleating head, preserving the set of recesses;
retracting the set of pleating heads radially outward, leaving the pleating tooth corresponding to each pleating head secured to the pleating nut;
moving a set of folding heads radially inwards, each folding head of the set of folding heads pushing a membrane material of the balloon inwards to define a plurality of folded portions; and
arranging the plurality of folded portions so that a first set of folded portions of the plurality of folded portions extend clockwise about a longitudinal axis of the delivery shaft and a second set of folded portions of the plurality of folded portions extend counterclockwise about the longitudinal axis of the delivery shaft.
